(19) United States
(12) Patent Application Publication
(10) Pub. No.: US 2025/0204080 A1
(43) Pub. Date: Jun. 19, 2025

(54) NANOPROJECTION ARRAY AND A METHOD FOR FABRICATING A NANOPROJECTION ARRAY

(71) Applicant: KING'S COLLEGE LONDON, London (GB)

(72) Inventors: Ciro Chiappini, London (GB); Cong Wang, London (GB); John McGrath, London (GB)

(21) Appl. No.: 18/849,261

(22) PCT Filed: Mar. 23, 2023
(86) PCT No.: PCT/GB2023/050733

§ 371 Date: Sep. 20, 2024

(30) Foreign Application Priority Data
Mar. 23, 2022 (GB) 2204062.0

(51) Int. Cl. H10F 77/162; B82Y 15/00; B82Y 20/00; B82Y 40/00; H10F 30/10; H10F 71/00

(52) U.S. Cl. CPC H10F 77/1625; B82Y 15/00; B82Y 20/00; B82Y 40/00; H10F 30/10; H10F 71/135

(57) ABSTRACT

The present disclosure relates to a method for fabricating an array of nanoprojections, where the nanoprojections may include nanopillars, nanowires, nanoneedles or nanocones. The present disclosure also relates to an array of the nanoprojections, and to uses of such arrays.

### CROSS REFERENCE TO RELATED APPLICATIONS

This is the U.S. National Stage of International Application No. PCT/GB2023/050733, filed Mar. 23, 2023, which was published in English under PCT Article 21(2), which in turn claims the benefit of Great Britain Application No. 2204062.0, filed Mar. 23, 2022, which is incorporated herein in its entirety.

### FIELD OF DISCLOSURE

The present disclosure relates to a method for fabricating an array of nanoprojections, where the nanoprojections may include nanopillars, nanowires, nanoneedles or nanocones. The present disclosure also relates to an array of the nanoprojections, and to uses of such arrays.

### BACKGROUND

Nanostructures have applications in many fields including electro-optical devices, sensors, and medical devices. Nanoprojections are nanostructures, and an array of nanoprojections is particularly effective at interacting with a biological system (e.g., a biological tissue) for biosampling, biosensing and drug delivery applications in the medical field. Conventionally, nanoprojections are manufactured from opaque and rigid semiconductors, which limits the functionality of the nanoprojection array when used in medical applications. For example, the opacity of the nanoprojection array makes it challenging to observe events occurring on the nanoprojections by optical microscopy techniques. Rigidity of an array of nanoprojections makes the application of the array to tissue less effective as the array cannot conform to the non-planar tissue surface. In particular, rigid arrays can easily break when deformed.

Many of the prior art arrays comprise non-porous (i.e., solid) nanoprojections. Such non-porous nanoprojections are typically non-degradable and have a limited capacity to absorb substances, e.g., molecules from a tissue sample, or therapeutic/diagnostic agents to be administered to a tissue.

Several approaches have been developed to form an array of nanoprojections on transparent and/or flexible substrates. One example approach, described in Kim et al., ACS Nano 14, 7227-7236 (2020) and Kim et al., Sci Adv. November 9; 4(11) (2018), focusses on developing an array of partially porous nanoprojections on flexible and/or transparent substrates. This approach has a complex way of detaching the array of nanoprojections from a first substrate in order to transfer the array to a second substrate—that is, the method requires the formation of undercuts to define the tips of the nanoprojection, where the tips of the nanoprojections are attached to a first substrate. The method further requires additional steps of spin coating the entire array of nanoprojections with a PVA or PDMS film which is cured and peeled for detaching the array of nanoprojections by applying a stress at the undercuts. A first drawback of this approach is that it requires several steps and a second drawback is that the application of stress at the undercuts can damage the tips of the nanoprojections. Moreover, the method has limitations on the aspect ratio and porosity of the resultant nanoprojections. For example, the above-mentioned undercuts used for detachment also define the tip size of the nanoprojections.

Weisse et al., (Nano Lett., 2013, 13, 4362-4368) and U.S. Pat. No. 10,037,896 disclose the transfer of silicon wire arrays using a single sacrificial porous silicon layer, and where the orientation of the silicon wire array is reversed on the final substrate.

The inventors have recognised the need for providing an improved method which enables the detachment of an array of nanoprojections from a first substrate in a simple manner so that the detached array can be transferred to a second substrate. The second substrate can be flexible and/or transparent. Such second substrates can have a desired flexibility, be non-toxic and have good cell and tissue compatibility.

### SUMMARY OF DISCLOSURE

The present disclosure relates to a method for fabricating an array of nanoprojections (e.g., nanocolumns, nanoneedles, nanowires and/or nanocones) from a first substrate. The method of the present disclosure configures the first substrate to enable the detachment of the array of nanoprojections from the first substrate in a simple manner. The detached array of nanoprojections can be transferred to a second substrate. The second substrate may be flexible and/or transparent.

The present disclosure also relates to a device comprising: an array of nanoprojections formed on a first porous layer, wherein the nanoprojections are at least partially porous; and a second substrate wherein the first porous layer is attached to the second substrate so that the first porous layer is adjacent to the second substrate and between the second substrate and the array of nanoprojections, and wherein the first porous layer and the second substrate are formed from different materials.

According to a first aspect of this disclosure, there is provided a method of forming an array of nanoprojections, the method comprising:
providing a first substrate;
performing a first etch of the first substrate to form an array of nanoprojections on the first substrate;
performing a second etch of the first substrate to form a first porous layer in the first substrate, wherein the first porous layer underlies the array of nanoprojections, and to either:
   (a) form a second porous layer in the first substrate, wherein the second porous layer underlies the first porous layer and has a higher porosity than the first porous layer such that the first porous layer is detachable from the second porous layer; or
   (b) remove a layer of the first substrate underlying the first porous layer so that the first porous layer is detachable from the first substrate;
attaching an adhesive layer over the top of the array comprising the nanoprojections so that the adhesive layer adheres to the free ends (i.e., tips) of the nanoprojections, and separating the adhesive layer, the array of nanoprojections and the first porous layer away from the second porous layer or the first substrate; and
attaching the array of nanoprojections and the first porous layer to a second substrate so that the first porous layer is adjacent to the second substrate and between the second substrate and the array of nanoprojections.

According to a further aspect of this disclosure, the method of forming the array of nanoprojections additionally comprises performing a third etch on the array of nanoprojections so that the nanoprojections are shaped as nanocones. The third etch can be performed at any point in the method. For example, the third etch can be performed before or after the first porous layer is detached from the second porous layer, or before or after the first porous layer is separated from the first substrate.

According to a further aspect of this disclosure there is provided a device comprising:
an array of nanoprojections formed on a first porous layer, wherein the nanoprojections are at least partially porous; and
a second substrate wherein the first porous layer is attached to the second substrate so that the first porous layer is adjacent to the second substrate and between the second substrate and the array of nanoprojections, and wherein the first porous layer and the second substrate are formed from different materials.

Further features and aspects of the disclosure are detailed below and defined in the appended claims.

### BRIEF DESCRIPTION OF THE DRAWINGS

The disclosure will now be described, by way of non-limiting examples, with reference to the accompanying drawings, in which:

FIGS. 1A-1E show the steps involved in a method for fabrication of nanoprojections on a porous substrate.

FIG. 2 shows a flow chart for the proposed fabrication method in FIGS. 1A-1E.

FIGS. 3A-3C show details of the steps involved in forming the nanoprojections, as well as two alternative ways for subsequently producing the nanoprojections arrays. FIG. 3A shows the use of MACE to form the nanoprojections. FIG. 3B shows an example of the subsequent processing steps, wherein electrochemical etching is used to form the first and second porous layers, the first porous layer is detached from the second porous layer, the nanoprojections formed on the first porous layer are transferred to a water-soluble tape, the first porous layer is transferred to a second substrate (i.e., a PDMS substrate), the water-soluble tape is dissolved, and RIE is used to shape the nanoprojections into nanocones. FIG. 3C shows an alternative example of the subsequent processing steps, wherein RIE is used to shape the nanoprojections into nanocones, electrochemical etching is used to form the first and second porous layers, the first porous layer is detached from the second porous layer, the nanoprojections formed on the first porous layer are transferred to a water-soluble tape, the first porous layer is transferred to a second substrate (i.e., a PDMS substrate), and the water-soluble tape is dissolved.

FIG. 4 shows an SEM image of a MACE fabricated nanoprojection (specifically nanocolumns/nanopillars) array.

FIGS. 5A-5E show various SEM images. FIG. 5A shows an array of nanoprojections (i.e., nanowires) as well as first and second porous layers. FIG. 5B shows the nanoprojection array formed on the first porous layer attached to a water-soluble tape, wherein the nanoprojections are shown extending downwards from the first porous layer and contacting the water-soluble tape. FIG. 5C shows the nanoprojection array (in this case nanocolumns) attached to the second substrate, namely a PDMS substrate. FIG. 5D shows the nanoprojection array (in this case nanocones) formed on the second substrate and wherein the first porous layer has been removed by etching. FIG. 5E shows the nanoprojection array attached to a flexible second substrate.

FIGS. 6A and 6B show SEM images of an array of nanoprojections (in this case nanocones) as well as the first and second porous layers. FIG. 6C shows an SEM image of an array of nanoprojections (in this case nanocolumns/nanopillars) as well as the first and second porous layers.

FIG. 7 shows an SEM image of a nanoprojection array (in this case nanocones) formed on a first porous layer attached to a water-soluble tape, wherein the nanoprojections are shown extending downwards from the first porous layer and contacting the water-soluble tape.

FIGS. 8A-8D provide various images of nanoprojections. FIG. 8A is a schematic image of a nanoprojection array (in this case nanocones). FIG. 8B is an SEM image of such an array.

FIG. 8C is an SEM image of nanoprojections having bulbous tips. FIG. 8D is an SEM image of nanopores formed on a nanoprojection.

FIG. 9 shows SEM images of various nanoprojection arrays formed on a PDMS substrate, wherein the lengths of the nanoprojections (in this case nanocones) differ due to the processes used in etching the nanoprojections. The heights range from about 1.5 to about 8 μm.

FIGS. 10A-10C show an image of a biodegradable gelatin hydrogel having a nanoprojection array formed thereon (FIG. 10A), as well as SEM images of the nanoprojection array formed on the biodegradable gelatin hydrogel (FIGS. 10B and C).

FIGS. 11A-11C show example applications of a nanoprojection array, formed using a method of this disclosure, for drug delivery.

FIGS. 12A-12B show a nanoprojection array formed on a curved tube. FIG. 12A shows the nanoprojection array on a curved tube, which is to be subsequently formed into a stent. FIG. 12B shows SEM images of the surface of curved tube showing the array of nanoprojections (in this case nanocones).

FIGS. 13A-13C show an image of a nanoprojection array formed on a substrate having an X-shaped cross section (FIG. 13A), as well as SEM images of the nanoprojection array formed on the substrate (FIGS. 13B and C).

FIG. 14 shows images of nanoprojection arrays on PDMS substrates, wherein the nanoprojection arrays have different thicknesses (Tₛ) of the first porous layer.

FIG. 15 shows the effect of reducing the thickness (Tₛ) of the first porous layer on transparency.

FIGS. 16A-16B show stretching of a nanoprojection array formed on a PDMS substrate from 100% to 180% (FIG. 16A), and an SEM image of the nanoprojection array following 50 cycles of stretching (FIG. 16B).

FIGS. 17A-17D show a stent having the nanoprojection array on its outer surface. FIG. 17A is a schematic image of the stent. FIG. 17B is an image of a stent produced by the method disclosed herein. FIG. 17C is an SEM image of the stent. FIG. 17D is an SEM image of the nanocone array present on the surface of the stent.

FIG. 18 shows a nanoprojection array, formed using a method of this disclosure, integrated with a stent and the use thereof.

FIGS. 19A-19C show contact lenses having a nanoprojection array formed thereon (FIG. 19A), and SEM images of the nanoprojection array formed on the contact lenses (FIGS. 19B and C).

FIGS. 20A-20C show images of the nanoprojection array applied to a wound dressing. FIG. 20A shows the nanoprojection array applied to a wound dressing. FIG. 20B is an SEM image showing the nanoprojection array and the wound dressing. FIG. 20C is an SEM image of the nanoprojection array (in this case nanocones) applied to the wound dressing.

FIGS. 21A-21E show an example application and corresponding results of a nanoneedle array used for molecular tissue sampling and the generation of molecular tissue replicas.

FIG. 22 shows SEM images of human dermal fibroblast cells seeded on a nanoprojection array at 2, 4 and 72 hours after seeding.

FIG. 23 shows confocal images of human dermal fibroblast cells seeded on a nanoprojection array at 2, 4 and 72 hours after seeding.

### DETAILED DESCRIPTION

The present disclosure relates to a method of forming an array of nanoprojections, the method comprising:
providing a first substrate;
performing a first etch of the first substrate to form an array of nanoprojections on the first substrate;
performing a second etch of the first substrate to form a first porous layer in the first substrate, wherein the first porous layer underlies the array of nanoprojections, and to either:
   (a) form a second porous layer in the first substrate, wherein the second porous layer underlies the first porous layer and has a higher porosity than the first porous layer such that the first porous layer is detachable from the second porous layer; or
   (b) remove a layer of the first substrate underlying the first porous layer so that the first porous layer is detachable from the first substrate;
attaching an adhesive layer over the top of the array comprising the nanoprojections so that the adhesive layer adheres to the free ends (i.e., tips) of the nanoprojections, and separating the adhesive layer, the array of nanoprojections and the first porous layer away from the second porous layer or the first substrate; and
attaching the array of nanoprojections and the first porous layer to a second substrate so that the first porous layer is adjacent to the second substrate and between the second substrate and the array of nanoprojections.

The method results in the transfer of the array of nanoprojections (e.g., nanocolumns, nanoneedles, nanowires or nanocones) and the first porous layer from the first substrate to a second substrate in a simple and efficient manner. In particular, the method ensures that the spatial arrangement of the nanoprojections on the first porous layer is maintained. Furthermore, the orientation of the nanoprojections is maintained, namely the free ends of the nanoprojections formed on the first substrate remain the free ends of the nanoprojections once the nanoprojection array has been transferred to the second substrate. Maintaining the orientation of the nanoprojections is particular important when the nanoprojections do not have a uniform width and/or structure. For example, when the nanoprojections are nanocones the width decreases towards the free ends, and to maintain such an orientation, it will be necessary to ensure that the orientation of the nanoprojections is maintained when transferring the nanoprojection array to the second substrate. Maintaining the correct orientation is also important when the porosity of the nanoprojections varies along the length of the nanoprojection. In the case of nanotubes and nanostraws, various openings can be positioned non-uniformly along the length of the nanotubes and nanostraws, e.g., in nanotubes the free end is generally open. It is therefore again important to maintain the correct orientation of the nanotubes and nanostraws once the array has been transferred to the second substrate.

According to certain embodiments, the method of forming the array of nanoprojections additionally comprises performing a third etch on the array of nanoprojections so that the nanoprojections are shaped as nanocones. The third etch can be performed at any point in the method. For example, the third etch can be performed before or after the first porous layer is separated from the second porous layer, or before or after the first layer is separated from the first substrate.

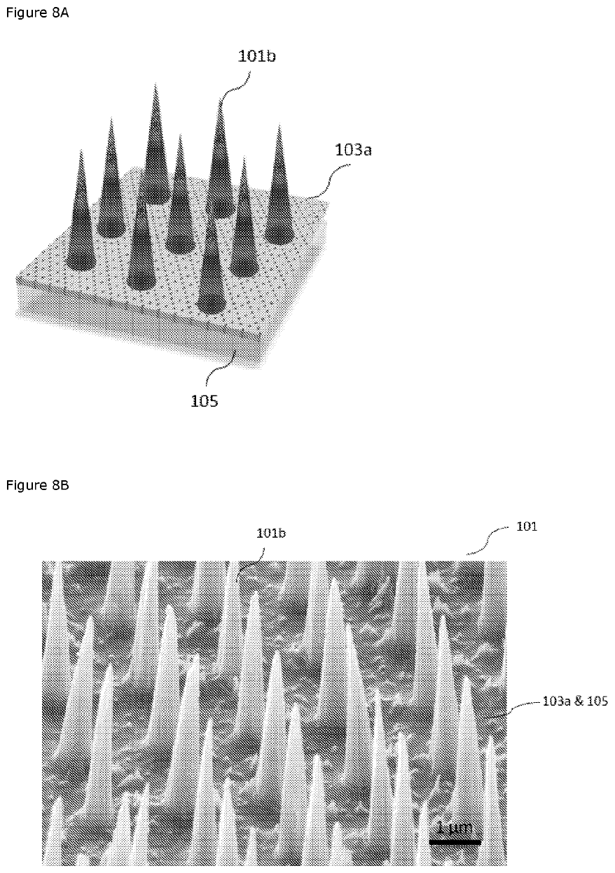

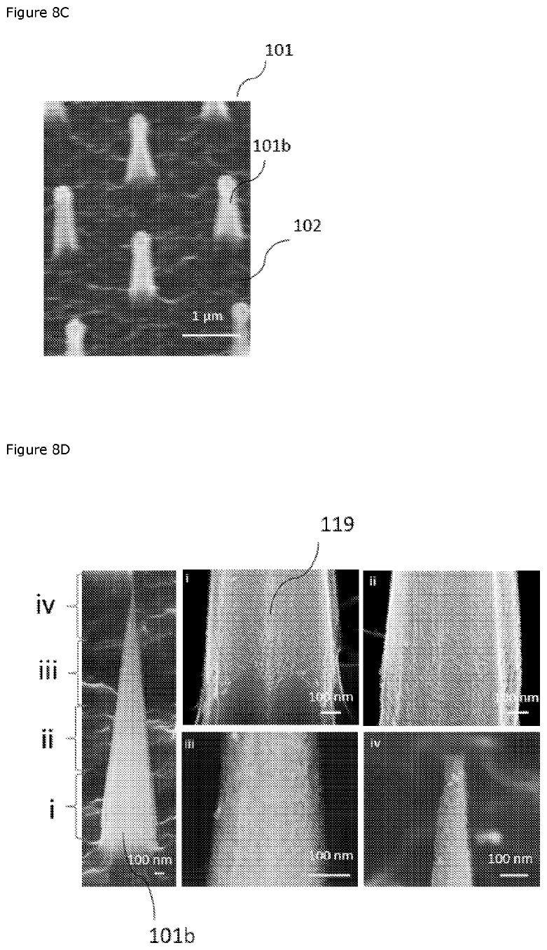

As utilized herein, the term “nanoprojection” refers to any three-dimensional high aspect ratio nanostructure (i.e., a structure having a larger vertical dimension than the lateral dimension) projecting from a surface of a substrate. The term “nanoprojection” encompasses structures including nanocolumns, nanoneedles, nanowires, nanotubes, nanostraws and nanocones. The terms “nanocolumns”, “nanoneedles” “nanowires”, “nanotubes” and “nanostraws” are terms used to describe substantially columnar structures having varying widths, and which may be solid, or in the case of nanotubes and nanostraws may have an internal bore. For example, nanocolumns have a relatively large width, nanowires have a relatively small width, and nanoneedles have an intermediate width. In certain embodiments, the nanostructure may have an approximately columnar or conical profile. In certain embodiments, the shape and/or length of the tip (i.e., the free end) may vary to control the sharpness of the tip. In certain embodiments, the nanoprojections may have a structure with a bulbous tip (e.g., a mushroom shape (see FIG. 8C).

In certain embodiments, the term “nanoprojection” refers to a high aspect ratio structure that is shaped and sized so that it can access the intracellular space of a cell.

The nanoprojection can be any suitable size. In some embodiments, the nanoprojection may be between about 0.5 and about 100 μm in length, between about 1 and about 100 μm in length, between about 1 and about 50 μm in length, between about 1 and about 15 μm in length, or between about 1 and about 10 μm in length. The nanoprojection in some embodiments may have a width of between about 1 and about 1000 nm, between about 20 nm and about 700 nm, or between about 50 and about 250 nm. As will be appreciated by those skilled in the art, the width of the nanoprojection may vary along the length of the nanoprojection. For example, when the nanoprojection is a nanocone, the width will decrease along the length of the nanocone towards the tip of the nanocone. The nanoprojections can be formed with different sized and shaped tips.

The term “nanocone” refers to a three-dimensional nanoprojection having an approximately conical profile, i.e., being an elongate structure with a tapered end. The tapered end assists the nanocone to penetrate a tissue (e.g., skin) and/or a cell.

The term “array” as used in this disclosure refers to any arrangement comprising a plurality of nanoprojections. In some embodiments, the array comprises 4 or more nanoprojections. In other embodiments the array comprises 9 or more nanoprojections. In some other embodiments, the array comprises over 100, over 1000 or over 10,000 nanoprojections. In the array, the nanoprojections can be arranged in any manner or pattern. For example, the nanoprojections may be evenly distributed across the array, may be distributed to have different densities across the array, or may be randomly arranged. Furthermore, the array may comprise substantially identical nanoprojections, or mixtures of different sized and shaped nanoprojections. In some embodiments, the array comprises substantially the same sized and shaped nanoprojections, e.g., the array may comprise only nanocones having substantially the same size. The array can have any density of nanoprojections. For example, the array may have a density of 1 nanoprojection every 200 nm² to 1 nanoprojection every 10,000 μm², 1 nanoprojection every 1 μm² to 1 nanoprojection every 1000 μm², 1 nanoprojection every 1 μm² to 1 nanoprojection every 625 μm², 1 nanoprojection every 2 μm² to 1 nanoprojection every 100 μm².

The term “first substrate” as used in this disclosure refers to the substrate from which the nanoprojections are formed. While the material for the first substrate is defined to be silicon in certain embodiments of this disclosure, any suitable semiconductor material known in the art can be used for the first substrate, including Germanium, Gallium arsenide, Gallium Nitride, silicon carbide, etc. In certain embodiments the first substrate is silicon.

The term “first etch” as used in this disclosure refers to any suitable process for etching a first substrate to form an array of nanoprojections on the first substrate. Suitable methods for performing such a first etch are well known to those skilled in the art.

A mask can be provided on the surface of the first substrate in a pattern which enables selective etching of the first substrate for forming the desired structures, i.e., nanoprojections. The mask comprises material that is resistant to etching and therefore protects (i.e., masks) areas of the first substrate from being etched. Suitable masks are well known to those skilled in the art. In some embodiments, the mask comprises an array of nanodots that are resistant to etching. The nanodots may be dielectric nanodots. As will be appreciated by those skilled in the art the mask can be in any suitable pattern depending on the arrangement, position, size and shape of the desired structures to be formed.

In certain embodiments, for example when using a metal-assisted chemical etching (MACE) process, areas of the first substrate that are to be etched, may have a metal layer deposited thereon. The metal layer can be deposited to form a desired pattern either by using a mask to prevent the metal layer coming into contact with the first substrate in areas that are not to be etched or by selectively depositing the metal layer so that it is only provided in areas that are to be etched. The application of such metal layers is well known to those skilled in the art.

In certain embodiments, the “first etch” may be any suitable wet-etch process known in the art for selective etching of the substrate material, such as a metal-assisted chemical etch process (MACE) electrochemical etching, stain etching or a combination thereof. In certain embodiments, the “first etch” is a MACE.

The term “second etch” as used in this disclosure refer to any suitable etching process for: (a) forming the first and second porous layers in the first substrate, wherein the second porous layer has a higher porosity than the first porous layer; or (b) forming the first porous layer and removing the layer of the first substrate underlying the first porous layer. For example, the second etch may comprise any known wet-etch process, such as MACE, electrochemical etching, stain etching or a combination thereof. The second etch may comprise a multi-step etch process for forming the first and second porous layers in the first substrate, or for forming the first porous layer and removing the underlying layer of the first substrate. In certain embodiments the second etch is used to forming the first and the second porous layers in the first substrate.

The “first etch” and “second etch” can be separate etches or can be part of a single multi-step etch process. Furthermore, the first and second etches can be performed in any order based on known processes in the art.

In certain embodiments, the second etch comprises an electrochemical etch which comprises a first etch step at a first set of bias conditions for forming the first porous layer in the first substrate, and a second etch step at a second set of bias conditions, to either:
(a) form the second porous layer in the first substrate; or
(b) remove the layer of the first substrate underlying the first porous layer.

In this context, the term “bias condition” refers to the parameters of the electrochemical etch, which include the bias current, bias voltage and the duration of the electrochemical etch. One or more of the parameters may differ between the first set and the second set of bias conditions. In one embodiment, the current and/or voltage, and the duration differ between the first set and the second set of bias conditions. In a further embodiment, the etchant used in the second etch step can be changed/varied between the first etch step and the second etch step to result in the different etching conditions. One skilled in the art will understand that it is possibly to change/vary the etchants used and/or the bias conditions to achieve the desired outcomes.

In certain embodiments where silicon is used as the first substrate, the second etch may be an electrochemical etch, wherein the etchant is an aqueous solution of hydrofluoric acid (HF). In some embodiments, the etchant may comprise an aqueous solution of HF including a tensioactive agent. The tensioactive agent may be an organic solvent such as ethanol (EtOH). The etchant may comprise an aqueous solution of HF and EtOH wherein the concentration of EtOH is greater than or equal to the concentration of HF, e.g., the ratio of HF to EtOH may lie within a range of 1:1 to 1:20, between 1:2 to 1:10 or between 1:3 to 1:5. The etchant may comprise an aqueous solution of HF and EtOH wherein the concentration of HF is greater than or equal to the concentration of EtOH, e.g., the ratio of EtOH to HF may lie within a range of 1:1 to 1:20, between 1:2 to 1:10 or between 1:3 to 1:5. Furthermore, in certain embodiments where a first and second porous layer are to be formed, for a given concentration of HF: EtOH, the first etch step and the second etch step may have a bias current density in a range between 0.01 mA/cm² to 1 A/cm² and a duration in a range between 1 ms to 600 s; however, the first etch step has a longer duration and/or a lower bias current density than the second etch step to result in the second porous layer having greater porosity that the first porous layer.

The first porous layer underlies the array of nanoprojections. The nanoprojections therefore extend from the planar surface of the first porous layer. When a second porous layer is formed, the second porous layer underlies the first porous layer so that the first porous layer and the second porous layer are adjacent to one another so that there is an interface between the layers.

The term “porous” as used herein refers to structures which have pores. The level of porosity is defined as the ratio of pore volume to the total volume of the structure.

The second porous layer has a higher porosity than the first porous layer such that the first porous layer is detachable from the second porous layer. A force can be applied to separate first porous layer from the second porous layer. In certain embodiments, the percentage increase in porosity of the second layer is at least 2%, at least 10%, at least 20% or at least 50% greater than the porosity of the first layer. Porosity of the first and second layers can be measured using, for example, the Brunauer-Emmett-Teller (BET) gas adsorption method known to those skilled in the art. In certain embodiments, the percentage porosity of the first layer can range between 1% to 90%, 1% to 80% or 1% to 50%. The absolute percentage porosity of the second layer can, in certain embodiments range between 3% to 95%, 3% to 85%, or 3% to 55%.

The formation of the first and second porous layers in the first substrate ensure that there is still a connection between the nanoprojection array and the first substrate thereby ensuring that the array of nanoprojections, including the first porous layer, is maintained in position on the first substrate and can only be removed by applying a force. The presence of first and second porous layers thereby avoids accidental loss or movement of the nanoprojection array from first substrate.

When a layer of the first substrate underlying the first porous layer is removed the first porous layer is no longer attached to the first substrate and can be easily removed from the first substrate.

The nanoprojections can be non-porous or porous. Porous nanoprojections can be partially porous, wherein only part of the nanoprojection is porous, (e.g., half of the nanoprojection is porous), or fully porous nanoprojections, wherein substantially the entire nanoprojection is porous, (e.g., between 80 and 100% or between 95 and 100% of the nanoprojection is porous). The nanoprojections can be made porous at any stage during the method of fabricating the array, e.g., during the first etch, the second etch or the third etch. The nanoprojections may have a uniform level of porosity or may have regions with different degrees of porosity. For example, the nanoprojections may have bands or regions of porosity alternating with and bands or regions of no porosity/lower porosity. In one embodiment, the nanoprojections may have a porous shell and a core of no porosity/lower porosity.

The first porous layer has the advantage of improving the flexibility and absorption capacity of the nanoprojection array while also enabling control of the biodegradability/solubility of the nanoprojection array in biological fluids. As indicated above, in certain embodiments the nanoprojections can be porous. Porous nanoprojections are advantageous as they have improved flexibility, biodegradability/solubility and have greater absorption/payload capacity compared to non-porous nanoneedle structures. In addition, when the porous nanoprojections are implemented together with the first porous layer, the overall absorption/payload capacity of the nanoprojection array is improved compared to using porous nanoprojections structures on a non-porous layer.

As indicated above, an adhesive layer is adhered to the free ends (i.e., tips) of the nanoprojections, and the adhesive layer, the array of nanoprojections and the first porous layer are separated away from the second porous layer or the first substrate. Any suitable method for separating the first porous layer from the second porous layer or the first substrate can be used. In embodiments where a first porous layer and a second porous layer are formed in the first substrate, the second porous layer is more porous than the first porous layer, and therefore the layers can be easily separated from one another. The method may comprise separating the first porous layer from the second porous layer or the first substrate by applying a force. The force applied to separate the first layer from the second layer or the first substrate may be any suitable force, including that achieved when physically peeling the adhesive layer, array of nanoprojections and the first porous layer away from the second porous layer or first substrate. In some embodiments, the force can be applied by means of ultrasonication or megasonication processes. In certain embodiments, the force may result in shattering the second porous layer or the first substrate, which will also result in separating the first layer.

In embodiments where a first porous layer and a second porous layer are formed in the first substrate, the porous layers are still connected together, and force will generally be required to separate the layers. In embodiments where the first substrate underlying the first porous layer has been removed the first porous layer is no longer connected to the first substrate and can therefore be easily removed.

In certain embodiments, the method may additionally comprise removing the adhesive layer after the first porous layer has been separated from the second porous layer or the first substrate. The adhesive layer can be removed in any manner. In certain embodiments, the adhesive layer is soluble, and can be removed using a suitable solubilising agent, e.g., water. Suitable adhesive layers include, for example, PVA-based adhesive tapes. In other embodiments, the adhesive layer can be UV-sensitive which loses adhesion when exposed to UV rays. In some other embodiments, the adhesive layer can be a temperature-sensitive adhesive which loses adhesion when heated.

As indicated above, once the first porous layer comprising the array of nanoprojections has been separated from the second porous layer or the first substrate, the array of nanoprojections and the first porous layer are attached to a second substrate so that the first porous layer is adjacent to the second substrate and between the second substrate and the array of nanoprojections.

The first porous layer can be subsequently removed using any suitable technique, such as an etching method, e.g., any of the etching methods referred to herein.

The second substrate can be any suitable substrate and differs from the first substrate, e.g., the second substrate is formed from a different material. The choice of the second substrate may depend on the intended application of the said array. In certain embodiments, the second substrate is a transparent and/or a flexible substrate. Suitable second substrates include hydrogel substrates, such as gelatin hydrogel substrates; polymers substrates such as silicones, e.g., poly(dimethylsiloxane) PDMS polymers, polylactic acid (PLA) polymers, polyvinyl alcohol (PVA) polymers, poly-L-lactic acid (PLLA) polymers, poly lactic-co-glycolic acid (PLGA) polymers, polycaprolactone (PCL) polymers, polystyrene (PS) polymers and photoresists; bandages such as wound contact layer bandages, wound dressings and silicone wound dressings; metal and metal oxide substrates, such as gold, silver, platinum, palladium, aluminium, aluminium oxide, indium, tin oxide; ceramic substrates; glass substrates; silicon substrates, such as silicon dioxide substates; quartz substrates; diamond substrates; and carbon substrates, such as graphene and graphite substrates. The second substrate may have any desired shape. For example, the substrate may be a planar substrate, a curved substrate or a lattice structure. The second substrate may also have a nanostructured surface and/or nanotopographies.

In a particular embodiment, the second substrate may form at least part of a medical device to come into contact with the body of a patient, e.g., a catheter, endoscopic probe or a stent, plaster, bandage, contact lens, etc. In a particular embodiment, the second surface is a surface of a stent, e.g., the outer surface of a stent. The array of nanoprojections and the first porous layer are thereby attached to a surface of the medical device; however, the first porous layer can be subsequently removed.

As indicated above, in certain embodiments, the method of forming an array of nanoprojections comprises performing a third etch on the array of nanoprojections so that the nanoprojections are shaped as nanocones. The third etch can be performed at any point in the method. For example, the third etch can be performed before separating the first porous layer from the second porous layer or the first substrate, or after the first porous layer has been separated from the second porous layer or first substrate.

For example, when the third etch is performed before the first porous layer is separated from the second porous layer/first substrate, the adhesive layer adheres to the free ends of the nanocones. When the third etch is performed after the first porous layer is separated from the second porous layer/first substrate, the third etch can be performed at any point after the first porous layer has been detached. For example, the method may comprise:
detaching the first porous layer with the array of nanoprojections from the second porous layer or the first substrate;
transferring the array of nanoprojections and the first porous layer to the second substrate; and
performing the third etch on the array of nanoprojections so that the nanoprojections are shaped as nanocones.

The third etch may be any suitable etch for shaping the nanoprojections as nanocones. Suitable etching methods for forming nanocones are well known to those skilled in the art. See, for example Chiappini et al., Nature Mater 14, 532-539 (2015). In certain embodiments, the third etch is a dry etch. Where the nanoprojections are silicon nanoprojections, the third etch may be a reactive-ion etch (RIE) process for etching the silicon nanoprojections in order to form silicon nanocones. RIE parameters such as the generator power, chamber pressure, gas precursors and duration of the etch can be tuned to provide nanocones having an average predetermined aspect ratio.

In certain embodiments, the nanoprojections (e.g., the nanocones) are biodegradable/soluble, i.e., the nanoprojections are absorbed in a biological environment, namely within the body of a patient or in a cell culture medium. The arrays of nanoprojections can be made biodegradable/soluble by ensuring that nanoprojections are porous. The greater the level of porosity the quicker the nanoprojections are biodegraded/solubilised.

The present disclosure also relates to an array of nanoprojections formed using the methods described herein.

The nanoprojection array of the present disclosure can be loaded with one or more therapeutic or diagnostic agents. The nanoprojection arrays can then be used to administer the therapeutic or diagnostic agents to cells and tissues. The cells and tissues may be in vitro or in vivo. The use of nanoprojections to administer diagnostic agents to cells and tissues is described in Chiappini et al., Nature Protocols, 16, 2021, 4539-4563. The nanoprojection arrays of the present disclosure can be formed on a flexible second substrate enabling the array to confirm to the contours of a target tissue. In addition, the porosity of the nanoprojections, as well as the first porous layer when present, can be controlled during the method of fabrication so that the amount of therapeutic or diagnostic agent to be absorbed, and therefore administered, can be controlled. The therapeutic agent can be any suitable agent including pharmaceutical agents, vaccines, antibodies, nucleic acid molecules, proteins, nanoparticles, etc. The diagnostic agent can be any suitable agent including labelled antibodies, dyes, radioactive markers, nanoparticles etc.

An additional aspect of the present disclosure is an array of nanoprojections fabricated using the methods described above and loaded with one or more therapeutic or diagnostic agents.

The present disclosure also relates to the use of an array of nanoprojections fabricated using the methods described above, and loaded with one or more therapeutic or diagnostic agents, for administering the one or more therapeutic or diagnostic agents to cells and/or a tissue, wherein the array of nanoprojections is placed in contact with the cells and/or tissue. The cells may be any cells to which it is desired to administer the therapeutic or diagnostic agents. The tissue may be any tissue (either within an organism or isolated from an organism, for example skin, to which it is desired to administer the therapeutic or diagnostic agents. In certain embodiments the one or more therapeutic or diagnostic agents are administered to cells and/or a tissue has been isolated from the human or animal body.

The nanoprojection array of the present disclosure can be used to obtain samples from cells and/or tissues. The cells and/or tissues can be in vivo or in vitro. In particular, the nanoprojections can be placed into contact with cells or a tissue and molecules from the cell or tissue will be absorbed by the nanoprojections. The molecules that can be absorbed include proteins, nucleic acids, lipids, metabolites, drugs, etc. The cells or tissue can be any suitable cells or tissue to which access can be obtained. For example, the cells may be part of a cell culture, and the tissue may be skin. The samples can be obtained in a simple and effective manner. Furthermore, as the spatial arrangement of the molecules in the cells or tissue is maintained on the nanoprojection array, a molecular replica is obtained indicating the spatial distribution of the molecules in the sample.

The finding that the spatial arrangement of the molecules is maintained on the nanoprojection array is advantageous as it provides an accurate indication of the distribution of the detected molecules in the cells or tissue.

The present disclosure also relates to the use of a nanoprojection array to obtain a molecular sample from cells or a tissue, wherein the nanoprojection array is placed in contact with the cells or the tissue so that molecules present within the cells or the tissue are absorbed onto the nanoprojections. The cells or tissue may be in vitro or in vivo. As the spatial arrangement of the molecules in the cells and tissue is maintained on the array a molecular cell/tissue replica can be obtained. The nanoprojection array can be any nanoprojection array. In certain embodiments the nanoprojections do not comprise any capture agents on their surfaces, e.g. antibodies or capture nucleic acid probe sequences. In other words, the nanoprojections are free of capture agents. In certain embodiments, the nanoprojections are porous. In certain embodiments, the nanoprojection array is obtained by the methods disclosed herein. In certain embodiments, the nanoprojection array is a nanoprojection array obtained by the methods disclosed herein wherein the second substrate is a transparent and/or a flexible substrate. The capacity of the nanoprojections to absorb the molecules can be increased by ensuring that the nanoprojections are porous. Furthermore, the use of a flexible second substrate will enable the nanoprojection array to flex and mirror the contours of the tissue being sampled. In addition, the use of a transparent second substrate will enable better subsequent measurement of the sampled molecules as methods involving optical detection techniques (e.g., fluorescence measurements) can be performed without the hinderance of an optically opaque substrate.

In certain embodiments, the use of a nanoprojection array to obtain a molecular sample from a tissue is performed on tissues that have been isolated from the human or animal body.

The present disclosure also relates to a medical device that comes into contact with the body of a patient, e.g., a catheter, endoscopic probe or a stent, plaster, bandage, contact lens, etc., wherein the medical device has a nanoprojection array fabricated using the methods described herein on at least part of its surface. In certain embodiments, the nanoprojection array is formed on a surface of the medical device that in use comes into contact with the body of a patient. The nanoprojection array can be formed on at least part of the surface of the medical device by adhering the first porous layer comprising the array of nanoprojections to at least part of the surface of the medical device. Accordingly, the surface of the medical device can function as the second substrate, or the nanoprojection array attached to different second substrate can be attached to a surface of the medical device. The adhesion can be achieved in any manner, including the use of surface functionalisation to ensure that the array adheres to the surface (e.g., using non-covalent (e.g., electrostatic interactions) or covalent bonding). Any adhesive can be used to attach the nanoprojection array to the surface of the medical device. Furthermore, the adhesion can be reversible. Numerous substrates have sufficient inherent adhesive properties to retain the porous layer. For example, hydrogels and PDMS substrates have sufficient inherent adhesive properties to retain the porous layer.

As will be appreciated by those skilled in the art, the nanoprojections may also be loaded with a desired therapeutic agent to achieve a desired therapeutic outcome, e.g., when the medical device is a stent, the therapeutic agent may reduce restenosis. Suitable therapeutic agents to reduce restenosis are well known to those skilled in the art, and include heparin, dexamethasone, sirolimus and paclitaxel. The nanoprojection array also provides improved fixation of the medical device with a tissue, e.g., with the vessel wall, thereby reducing the risk of the medical device moving once inserted. In certain embodiments, the medical device is a stent.

The present disclosure also relates to a device comprising:
an array of nanoprojections formed on a first porous layer, wherein the nanoprojections are at least partially porous; and
a second substrate wherein first porous layer is attached to the second substrate so that the first porous layer is adjacent to the second substrate and between the second substrate and the array of nanoprojections, and wherein the first porous layer and the second substrate are formed from different materials.

The second substrate can be any suitable substrate having any shape, as described above. In certain embodiments the nanoprojections of the device may be loaded with one or more therapeutic or diagnostic agents as discussed above.

The present disclosure also relates to a method of forming an array of nanoprojections, the method comprising:
providing a first substrate;
performing a first etch of the first substrate to form an array of nanoprojections on the first substrate;
performing a second etch of the first substrate to form a first porous layer in the first substrate, wherein the first porous layer underlies the array of nanoprojections, and forming a second porous layer in the first substrate, wherein the second porous layer underlies the first porous layer and has a higher porosity than the first porous layer such that the first porous layer is detachable from the second porous layer.

This method of forming the array of nanoprojections may additionally comprise separating the first porous layer from the second porous layer. Methods for separating the first porous layer from the second porous layer are as defined above.

This method may also additionally comprise attaching the separated array of nanoprojections and the first porous layer to a second substrate. Methods for attaching the separated array of nanoprojections and the first porous layer to a second substrate are as defined above. The second substrate is also as defined above.

This method may also additionally comprise performing a third etch to shape the nanoprojections into nanocones. The step of performing the third etch is as defined above.

The present disclosure will now be described with reference to the attached figures.

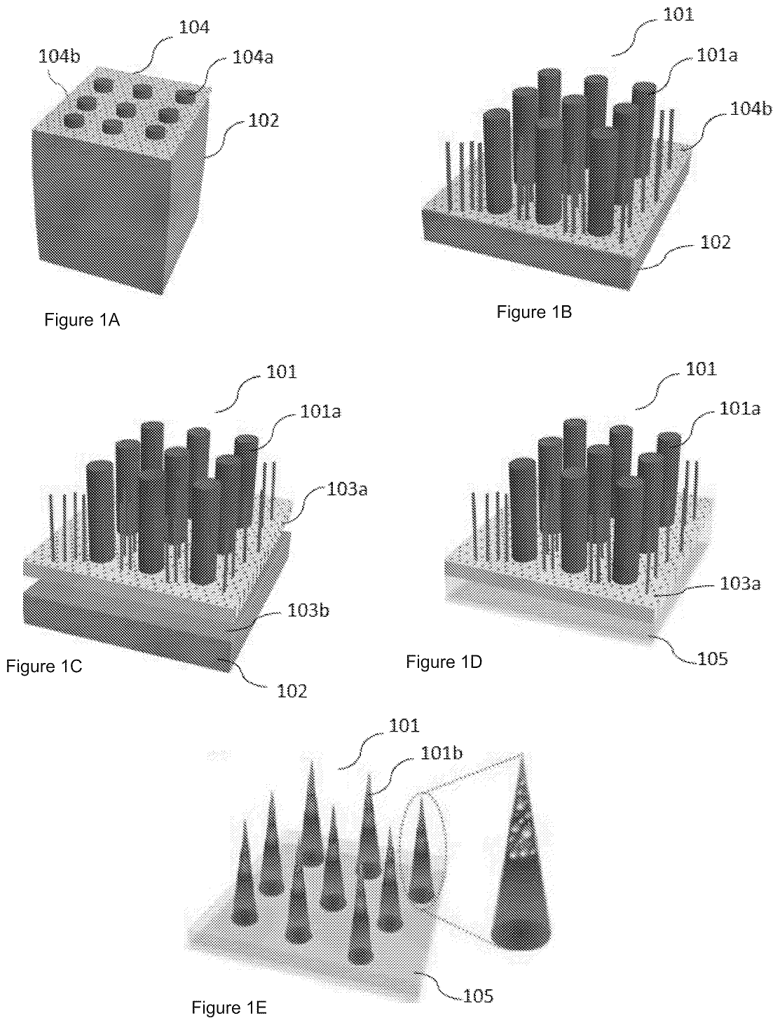

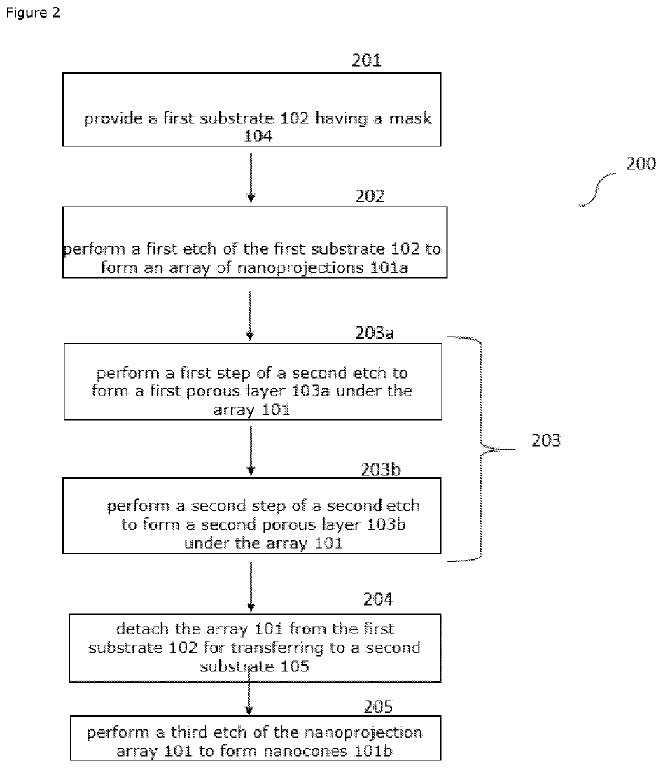

FIGS. 1A-1E show a diagrammatic representation of the steps involved in a method of fabricating an array of nanonprojections, according to an embodiment of this disclosure. FIG. 2 shows a corresponding flow-chart 200 of the steps involved in the method of FIGS. 1A-1E.

As can be seen in FIG. 1A, a first substrate 102 (in this example a silicon (Si) wafer, although other substrates may be used) is provided with a mask 104 for forming an array 101 of nanoprojections (in this example nanopillars) 101a (FIG. 1A, Step 201 of FIG. 2). The mask 104 comprises an array of nanodots 104a, where the nanodots can be dielectric nanodots. Any suitable patterning means can be used for forming the mask, such as photolithography, nanosphere lithography or electron beam lithography. A metal layer 104b is also deposited on the top surface of the first substrate 102, while excluding the array of nanodots 104a.

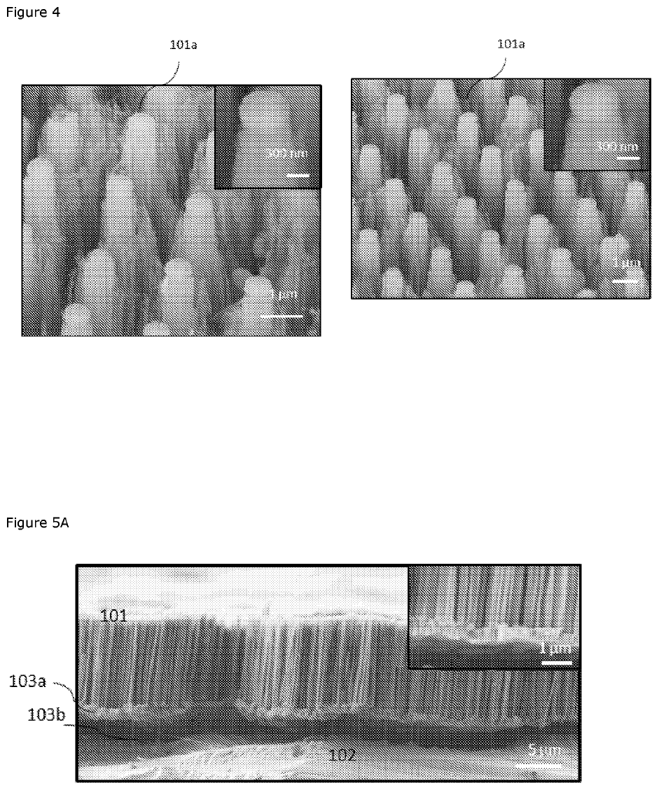

A first etch is performed on the first substrate 102 having the mask 104 to form an array 101 of nanopillars 101a (FIG. 1B, Step 102 of FIG. 2). In the example of FIGS. 1A-1E, the first etch comprises a wet-etch metal-assisted chemical etch (MACE) process that selectively etches the first substrate covered by the metal layer 104b to form the array 101 of nanopillars 101a. The residual metal layer 104b on the top surface of the first substrate is removed after the completion of the first etch. FIG. 4 shows an SEM image of nanopillars produced using a MACE process.

The use of MACE, as described above for the first etch, may result in the formation of porous silicon nanoprojections. U.S. Pat. No. 8,568,877 B2, the entirety of which is incorporated herein by reference, describes how the porosity and pore size of porous silicon nanoprojections, formed using the MACE process, can be dependent on at least one of the doping of the silicon wafer, the resistivity of the wafer, the type of metal in the metal layer and concentration of chemicals and solvent in the etchant used for the wet etch. Chiappini et al., (Adv. Func. Mater., 2010, 20, 2231-2239) and Handbook of Porous Silicon (Mace Silicon Nanostructures, Chiappini, pp 247-267, 2018), both of which are incorporated herein by reference further describes how the desired shape, porosity and pore size of porous silicon nanoprojections can be obtained using etching processes.

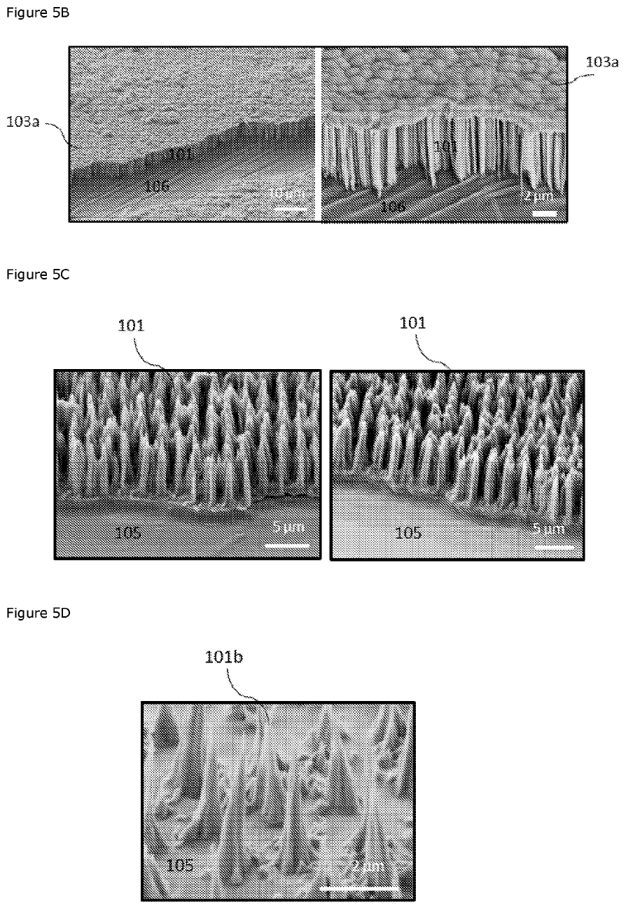

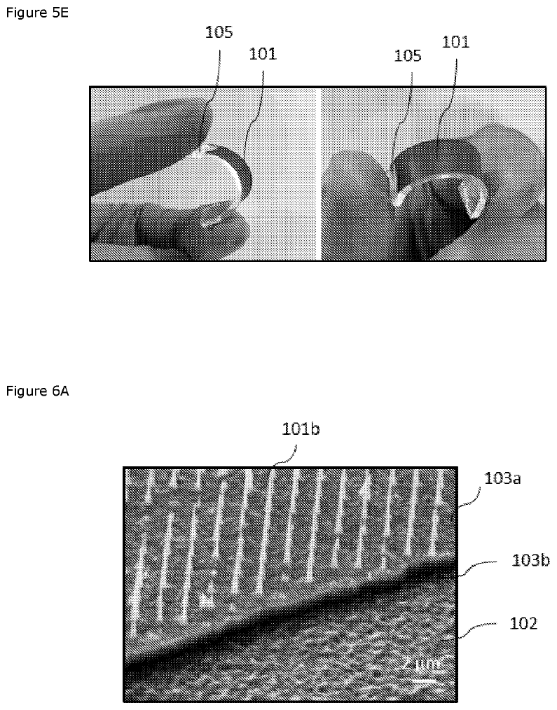

A second etch is performed on the first substrate 102, to form first and second porous layers, 103a and 103b (also referred to herein as the “porous layer” and the “detachment layer”, respectively). In the example of FIG. 1C, where the first substrate is a silicon wafer, the second etch comprises a multi-step electrochemical wet-etch process. In particular, the multi-step electrochemical wet-etch process comprised a first etch step for forming the first porous layer, and a second etch step for forming the second porous layer. An etchant comprising a ratio of HF: EtOH at 1:3 was used for both the first and second etching steps. The electrode in solution was platinum, and the electrode on the other side of the silicon wafer was aluminium. The first etch step was performed for a duration of 60 s at 2 A to form the first porous layer 103a under the nanopillar array. The second etch was then performed for a duration of 2 s at 6 A to form the second porous layer 103b below the first porous layer 103a. As will be appreciated by those skilled in the art any suitable conditions, etchants, electrodes can be used to form the first and second porous layers. The second porous layer 103b has a higher porosity than the first porous layer 103a. An array 101 of nanoprojections 101a with the first 103a and second porous layers 103b formed using the above-mentioned electrochemical etch process is shown in the SEM image of FIG. 5A. In FIG. 5A the nanoprojections are nanowires; however, as will be appreciated by those skilled in the art the nanonprojections can be any desired form.

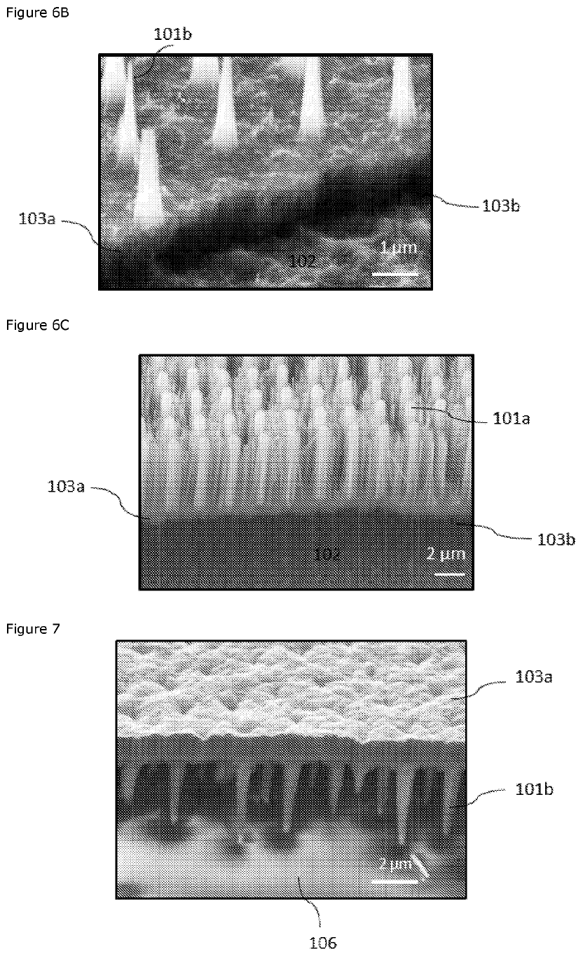

The second etch results in the formation of a first porous layer 103a directly under and contiguous with the nanopillar array 101 and a second porous layer 103b below the first porous layer 103a such that the second porous layer 103b is formed between the first porous layer 103a and the non-etched first substrate 102. See, for example, FIG. 1C, FIG. 5A, FIG. 6A, FIG. 6B and 6C, which show such an arrangement. Step 203a of FIG. 2 is a first etch step of a multi-step etch process 203 for forming a first porous layer 103a under the nanopillar array 101. Step 203b of FIG. 2 is a second etch step of a multi-step etch process 203 for forming a second porous layer 103b between the first porous layer 103a and the non-etched first substrate 102. The second etch is configured such that the second porous layer 103b is of a higher porosity than the first porous layer 103a.

The higher porosity of the second porous layer 103b enables the second porous layer 103b to function as a detachment layer. That is, the nanoprojection array 101, together with the first porous layer 103a, can be detached upon application of a force at the interface between the first porous layer 103a and the second porous layer 103b. The detached nanoprojection array 101 may then be transferred onto a second substrate 105 (FIG. 1D, Step 204 of FIG. 2).

The nanoprojection array and the first porous layer are separated from the second porous layer using a water-soluble adhesive layer (also referred to herein as a water-soluble tape) and is discussed in further detail below.

While FIG. 1D shows the nanoprojection array 101 on the second substrate 105 with the first porous layer 103a, the first porous layer can be etched away. See FIG. 1E, where the first porous layer has been etched away. In some embodiments, the first porous layer 103a is retained and in other embodiments the first porous layer is removed.

The nanoprojections are etched further (using a third etch) to shape them into nanocones, which may be porous, as will be explained in more detail below with reference to FIG. 1E. The use of an array of porous nanoprojections (e.g., nanocones), optionally together with a first porous layer 103a under the array, advantageously improves the overall absorption capacity of the array of nanoprojections when used, for example, in drug delivery applications or when taking a sample from a tissue.

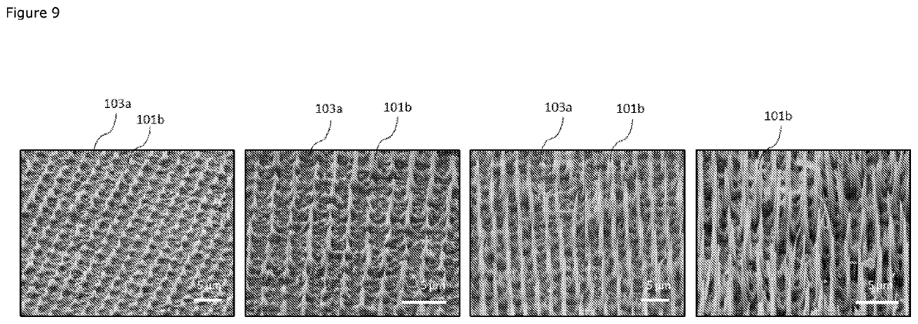

Nanoprojections, especially nanocones, are advantageous for medical applications which require minimal invasive interfacing with biological systems. In the example of FIG. 1E, the array 101 of silicon nanoprojections 101a are etched using a dry-etch process, in particular, a reactive-ion etching (RIE), to shape the nanoprojections into nanocones 101b; however, any suitable dry-etch process can be used for performing the third etch. The RIE parameters used were SF₆ (20 sccm), 7 min, 300 W (forward), 100 mTorr, 100 strike pressure. RIE parameters, such as the generator power, chamber pressure, etching gas formulation and its partial pressure, and duration of the etch, can be tuned to adjust the aspect ratio of nanocones 101b. FIGS. 5C and 5D, and FIGS. 8A-8D show SEM images of an array of silicon nanocones formed by an RIE etch. Furthermore, FIG. 9 shows different arrays of nanocones, wherein the length of the nanocones has been changed by varying the RIE parameters used to shape the nanoprojections. The nanocones have lengths varying from about 1.5 to about 8 μm.

The nanoprojections can be shaped into nanocones at any point in the process of manufacturing the nanoprojection array. In particular, the nanoprojections can be shaped into nanocones after the nanoprojection array has been transferred to a second substrate or before such a transfer. In FIG. 2, the array 101 of nanoprojections 101a can be etched to form an array of nanocones 101b after the array 101 of nanoprojections 101a is detached and transferred onto the second substrate 105 (step 204 of FIG. 2). However, this is just one embodiment of the method. The nanoprojections can be shaped to form the array of nanocones before or after transfer to a second substrate.

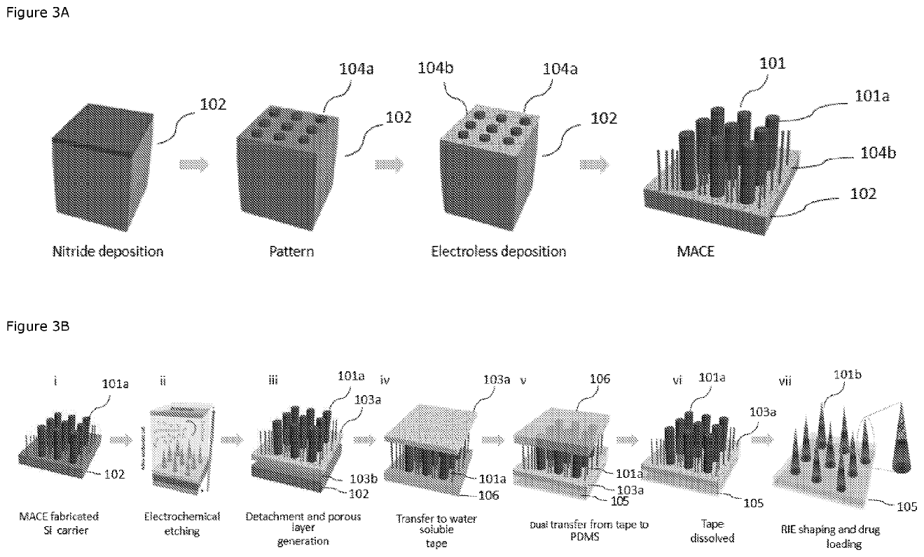

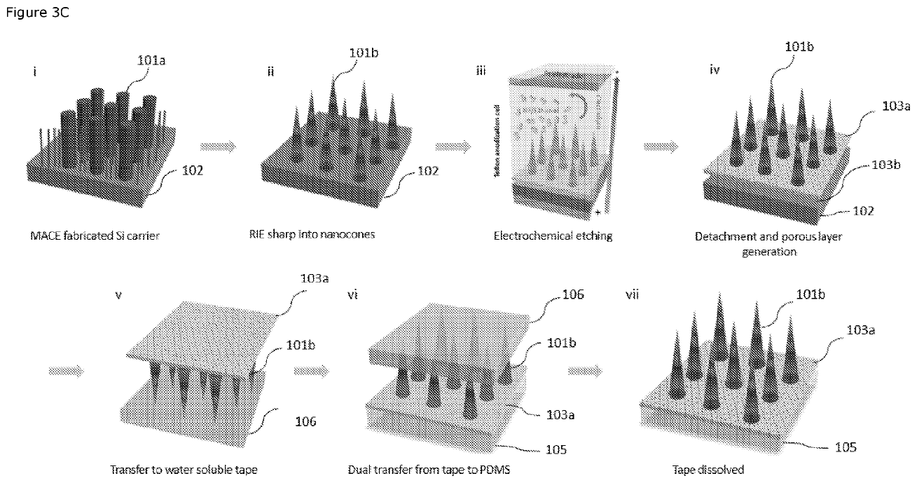

In FIG. 3A, the MACE process of forming the nanoprojections 101a is shown in the same manner as described with respect to steps (a) and (b) of FIGS. 1A-1E. FIG. 3B then shows how, according to one embodiment, an electrochemical etch is performed to form the first and second porous layers 103a, 103b, and the first porous layer 103a is detached from the second porous layer 103b and transferred to a water-soluble tape 106. The nanoprojection array 101 (including the first porous layer 103a) is then transferred to a second substrate 105, namely a PDMS substrate (but any suitable substrate could be used). The tape 106 is then dissolved and at this stage the nanoprojections 101a are shaped into nanocones 101b. At the same time as forming the nanocones 101b, the first porous layer 103a can be removed (or made thinner, or made more porous) by the same etching process used to shape the nanoprojections 101a or by using a different etching process. The shaped nanocones 101b can also be loaded with a therapeutic agent or diagnostic agent to be administered to cells or a tissue using the array 101.

As an alternative to the embodiment shown in FIG. 3B, FIG. 3C shows that after the MACE process of forming the nanoprojections 101a shown in FIG. 3A, the nanoprojections are shaped into nanocones 101b. Thereafter an electrochemical etch is performed to form the first and second porous layer 103a, 103b, the first porous layer 103b is detached from the second porous layer 103a and transferred to a water-soluble tape 106. The nanoprojection array 101 (including the first porous layer 103a is then transferred to a second substrate 105, namely a PDMS substrate (but any suitable substrate could be used). The tape 106 is then dissolved leaving the array of nanocones 101b and the first porous layer 103a on the second substrate 105. The first porous layer 103a can be removed (or made thinner, or made more porous) at this stage by using any suitable etching process. The shaped nanocones 101b can also be loaded with a therapeutic agent or diagnostic agent to be administered to cells or a tissue using the array. This embodiment of shaping the nanoprojections 101a into nanocones 101b prior to any transfer to a second substrate 105 may be desired if the second substrate 105, onto which the array 101 is to be transferred, is not compatible with the etching process used to shape the nanoprojections 101a into nanocones 101b, e.g., a dry-etch process such as RIE. For example, a hydrogel substrate or a curved (non-flat) substrate is not generally compatible for use in a RIE etch—in this case, the array 101 of nanoprojections 101a can be shaped using RIE before the array 101 is transferred to the hydrogel or curved substrate.

As indicated above, the array 101 of nanoprojections 101a, 101b with the first porous layer 103a, is detached using a water-soluble adhesive layer 106 (also referred to herein as a “tape”). The water-soluble adhesive layer 106 is attached to the nanoprojections 101a, 101b in the array 101. The array 101, together with the first porous layer 103a, can then be physically detached from the second porous layer 103b by peeling the adhesive layer 106 so as to apply a force at the interface between the first porous layer 103a and the second porous layer 103b. The array 101 together with the first porous layer 103a can then be transferred to a second substrate 105. FIG. 5A shows an array 101 of nanoprojections 101a (in this case nanowires), the first porous layer 103a, the second porous layer 103b and the non-etched base silicon substrate 102. FIG. 5B shows the array 101 wherein the nanoprojection array and the first porous layer 103a have been detached and are adhered to water-soluble adhesive layer 106. While this example describes the transfer process using a water-soluble adhesive tape 106 as the adhesive layer, any other suitable adhesive layer can be used for the transfer process.

FIGS. 6A and 6B shows an array 101 of nanoprojections (in this case nanocones 101b), the first porous layer 103a and the second porous layer 103b. FIG. 6C shows an array 101 of nanoprojections (in this case nanocolumns 101a), the first porous layer 103a and the second porous layer 103b. The array 101 and the first porous layer 103a can be separated from the second porous layer 103b by adhering the free ends of the nanoprojections 101a, 101b to a water-soluble adhesive layer 106 and then peel the layers apart. FIG. 7 shows the nanoprojection array 101 and the first porous layer 103a adhered to the water-soluble adhesive layer 106 after separation from the second porous layer 103b.

The nanoprojection array 101 and the first porous layer 103a is then transferred onto a PDMS substrate 105. The water-soluble adhesive layer 106 can be dissolved in water once the transfer of the array 101 and the first porous layer 103a onto the second substrate 105 is complete. The first porous layer 103a can be bonded to the PDMS substrate 105 using any suitable means known to the person skilled in the art. For example, due to the inherent chemistry of the silicon first porous layer 103a and the PDMS substrate 105, a bond will form. The strength of the bond could be increased using techniques known to those skilled in the art, including the use of oxygen plasma. As will be appreciated by those skilled in the art any suitable method for forming the bond can be used. For example, the surface of the second substrate 105 can be made either electrostatically attractive to the first porous layer 103a of the nanoprojection array 101 or reactive for covalent bonding. Numerous ways are known in the art for functionalising a surface with adhesive molecules. Other methods of applying a suitable adhesive agent to one or both of the first porous layer 103a and the second substrate 105 to enable a bond to be formed are well known to those skilled in the art. FIG. 5C and FIGS. 8A-8D show a nanoprojection array 101 and a first porous layer 103a attached to a PDMS substrate 105. FIG. 5E also shows a nanoprojection array attached to a flexible PDMS substrate.

The transparency of the nanoprojection array can be controlled by changing the thickness of the first porous layer 103a (defined as Tₛ). By having a relatively transparent nanoprojection array there are greater opportunities for exploring real-time interactions and to track dynamic cellular behaviour, which would be limited by having an opaque structure. Nanoprojection arrays were formed from a Si wafer (i.e., the first substrate). As indicated above, a first etch is performed to form the array of nanoprojections. A second etch is performed to form the first porous layer 103a and the second porous layer 103b. With respect to the second etch, a first etch step is used to form the first porous layer 103a and a second etch step is used to form the second porous layer 103b. The first etch step was performed using a 1:1 (v/v) mixed electrolyte solution of 50% HF (130 ml) and 99% ethanol (130 ml), with a current density of 8.4 mA cm⁻² for 60 s to generate a 600 nm thick first porous layer. The second etch step was performed using a 1:3 (v/v) mixed electrolyte solution of 50% HF (65 ml) and 99% ethanol (195 ml) at 101 mA cm⁻² for 2 s to form the second porous layer 103b.

The nanoprojection array 101 and first porous layer 103a are detached from the second porous layer 103b and transferred to a PDMS substrate (i.e., the second substrate) using a water-soluble tape, as described above. The water-soluble tape 106 was then dissolved.

The transferred nanoprojection array has a 600 nm thick first porous layer 103a.

To reduce the thickness of the first porous layer 103a, the transferred nanoprojection array is subjected to RIE in SF₆ (20 sccm) plasma at 200 W, 100 mTorr, 100 strike pressure for 150 s to reduce the thickness of the first porous layer 103a to 200 nm.

The 80 nm thick layer was formed by subjecting the transferred nanoprojection array to multi-RIE steps in SF₆ (20 sccm) plasma at 300 W, 100 mTorr, 100 strike pressure for 90 s, followed by SF₆ (20 sccm) plasma at 200 W, 100 mTorr, 100 strike pressure for 40 s, followed by SF₆ (20 sccm) plasma at 200 W, 10 mTorr, 50 strike pressure for 90 s.

The 0 nm layer (i.e., no first porous layer 103a) was formed by taking the transferred nanoprojection array reducing the first porous layer 103a to 80 nm by using the multi-RIE steps described above, and then additionally performing a RIE step of SF₆ (20 sccm) plasma at 200 W, 10 mTorr, 50 strike pressure to remove the first porous layer.

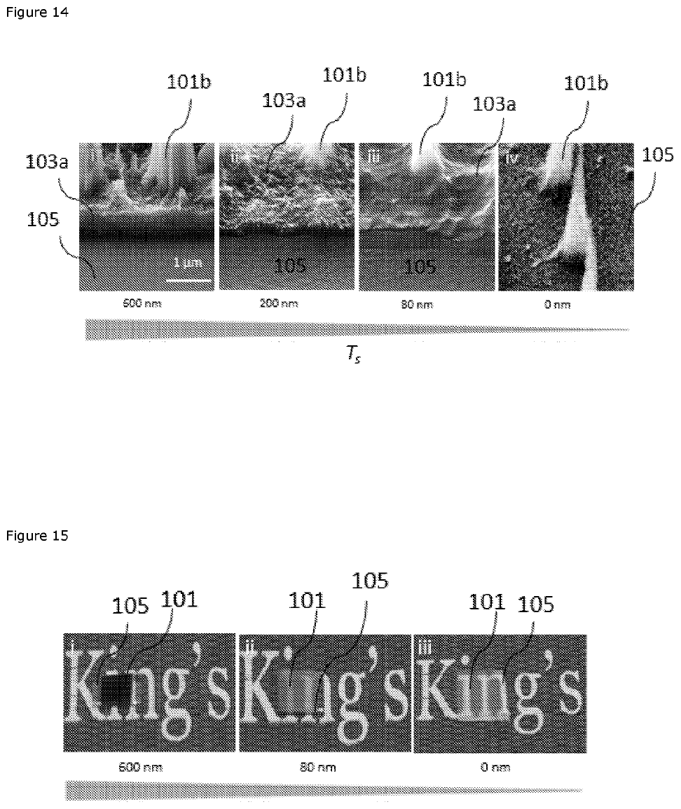

FIG. 14 shows the interfaces between the nanoprojection arrays and the PDMS second substrate (105) on which the nanoprojection arrays are attached. The thickness of the first porous layer (103a) varies in each image: image (i) thickness of the first porous layer (103a) is 600 nm; image (ii) thickness of the first porous layer (103a) is 200 nm; image (iii) thickness of the first porous layer (103a) is 80 nm; and image (iv) thickness of the first porous layer (103a) is 0 nm.

FIG. 15 shows how differences in the thickness of the first porous layer enable the transparency of the structure to be varied from opaque when the thickness of the first porous layer (103a) is 600 nm to transparent when thickness of the first porous layer (103a) is 0 nm.

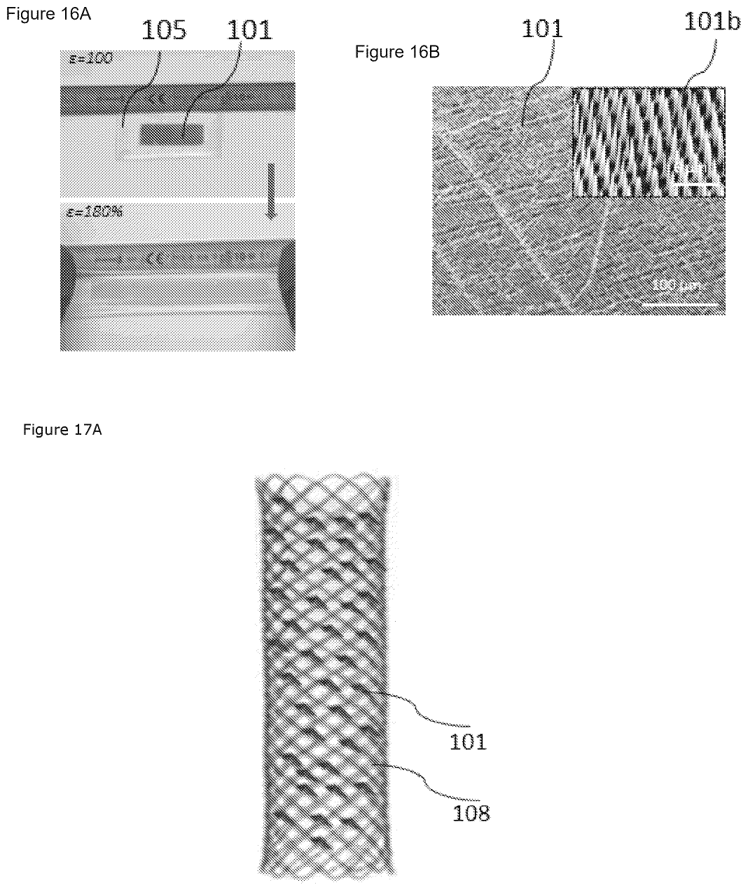

FIGS. 16A-16B show that the nanoprojection array (having a 600 nm thick porous layer (103a)) formed on the PDMS second substrate can be stretched to at least 180% of its original size. See FIG. 16A. FIG. 16B shows that after 50 cycles of stretching the nanoprojection array formed on the PDMS second substrate the structure of the nanoneedle array remains intact.

FIGS. 5D, 8 and 9 show examples of arrays 101 of nanocones 101b on the second substrate 105, according to an embodiment of this disclosure. The array 101 of nanocones 101b is formed by etching an array of nanopillars 101a as described above. The nanocones 101b in the array 101 can be of any desired length and any desired porosity. The length and porosity of the nanocones 101b can be determined by controlling the etching parameters. In FIG. 9, different lengths of the nanocones 101b are produced. Furthermore, the porosity of the nanocones 101b can be varied by controlling the etching conditions. The nanoncones 101b can be partially porous (e.g., on parts of the nanoncones 101b, such as the tips, are made porous), and in other embodiments they may be fully porous (e.g., the nanocones 101b are porous along substantially their entire length). FIG. 8C shows that the nanoprojections 101b can be formed with bulbous tips. Furthermore, FIG. 8D shows how a nanocone (101b) can have formed with nanopores (119) along the length of the nanocone, and that the nanopores can be formed to modify the porosity of the nanoprojection.

Although not shown in the Figures, the methods described above and shown in FIGS. 3A to 3C can be adapted so that the second etch forms a first porous layer in the first substrate and removes a layer of the first substrate underlying the first porous layer. In such a situation an adhesive layer is again adhered to the free ends (i.e., tips) of the nanoprojections, and the adhesive layer, the array of nanoprojections and the first porous layer separated away from the first substrate. Thereafter, the nanoprojection array 101 (including the first porous layer 103a) is then transferred to a second substrate 105 in the same manner as shown in FIGS. 3B and 3C.

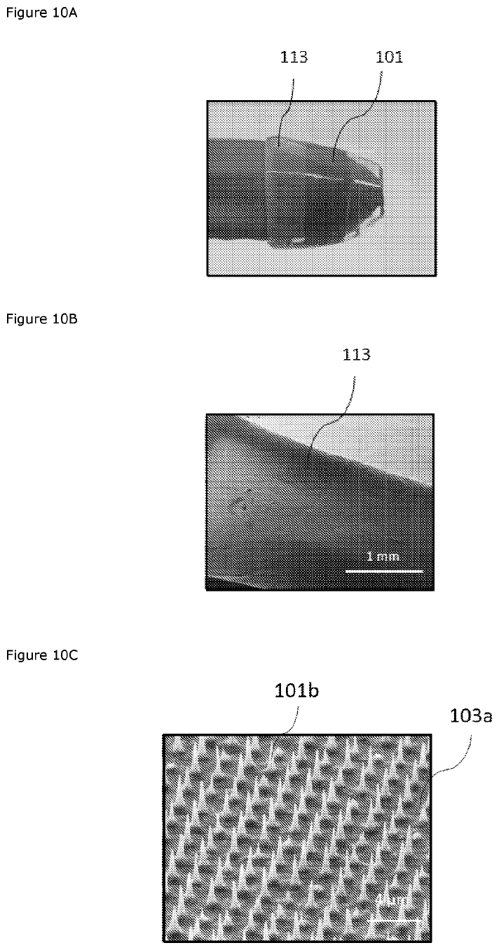

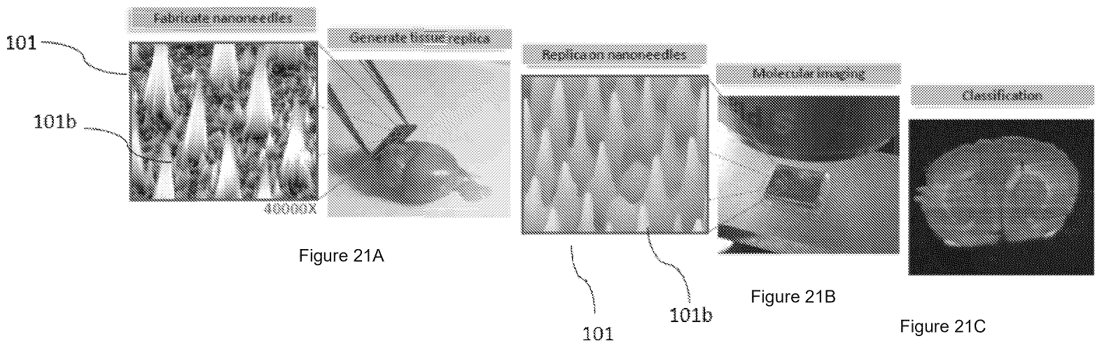

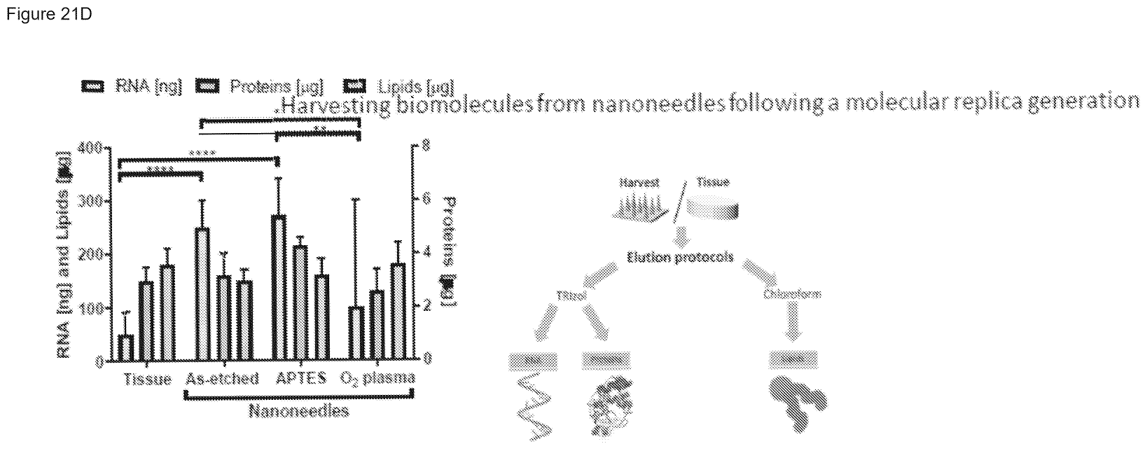

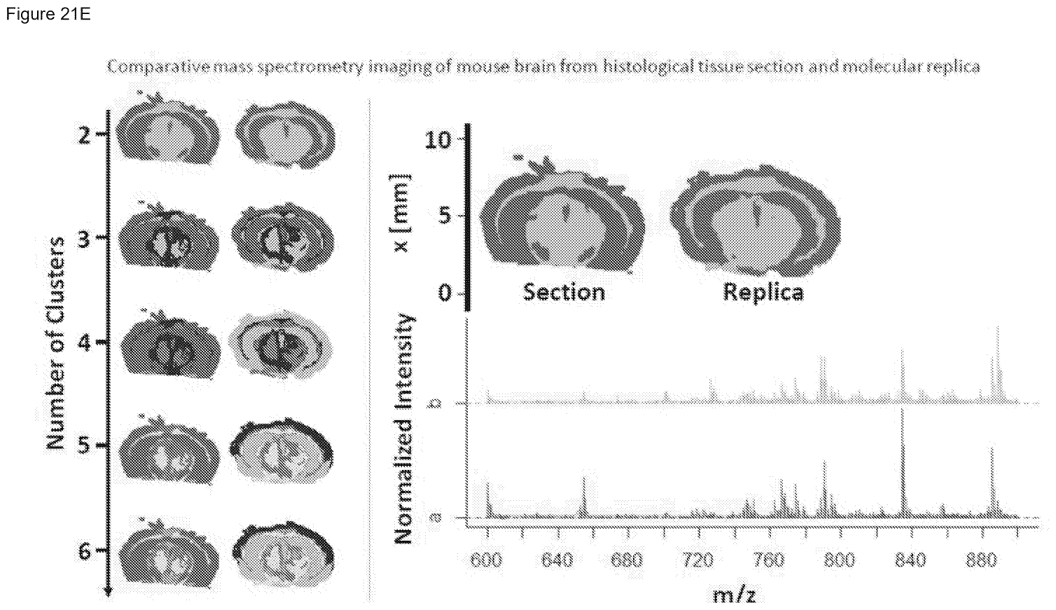

Example applications of a nanoprojection array 101 will be now described with reference to FIGS. 10-22.

### Drug Delivery and other Biomedical Applications

In FIGS. 10A-10C, a nanoprojection array is provided on a flexible bio-degradable gelatin hydrogel (113). The gelatin hydrogel functions as the second substrate (105) and is an efficient platform for tissue engineering and biomedical applications, especially wherein degradation of both the nanoprojections and substrate is desired.

Gelatin (from porcine skin) was dissolved in deionized water at 50° C. to obtain a 10 wt % solution. The crosslinkers EDC (1-Ethyl-3-(3-dimethylaminopropyl)carbodiimide) and NHS (n-hydroxysuccinimide) were separately dissolved in deionized water to obtain 1 M and 0.4 M stock solutions, respectively. The EDC and NHS solutions were sterilized through 0.22 μm filters and added to gelatin solution to achieve 25 mM of EDC and 10 mM of NHS concentration in the final solution. The crosslinked solution was then poured into a petri dish and incubated at 37° C. until gelled. The nanoprojection array (101) was then transferred to the gelatin hydrogel (113) via a water-soluble layer (106) as described above. The water-soluble layer was dissolved leaving the nanoprojection array (101) attached to the hydrogel (113). See FIG. 10A wherein the gelatin hydrogel having the attached nanoprojection array is shown. FIGS. 10B and 10C show SEM images of the nanoprojection array on the biodegradable gelatin hydrogel at different magnifications.

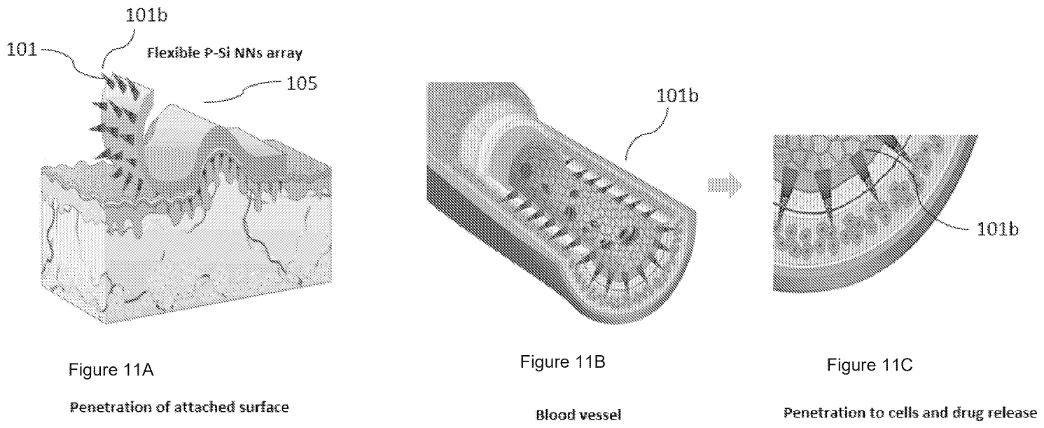

FIGS. 11A-11C show an example use of a nanoprojection array 101 formed using the method disclosed herein for drug delivery application. The nanoprojection array 101 is provided on a second substrate 105, which in this example, is configured to be a flexible substrate which enables the array to conform to the contours of the tissue. The nanonprojections (in this case nanocones 101b) have been pre-loaded with a therapeutic agent. The profile of the nanoprojections 101b enables the nanoprojections 101b to penetrate the tissue surface and to deliver a therapeutic agent to the cells of the tissue.

### Stents and other Structures

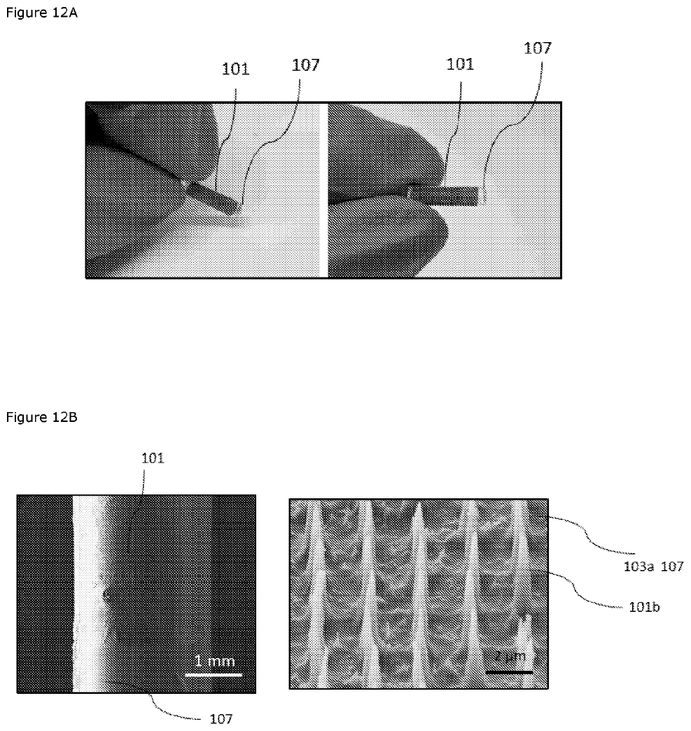

FIGS. 12A and 12B show shows a nanoprojection array 101 formed on a curved tube 107.

A curved tube 107 (stent tube), which is subsequently to be formed into a stent 108, acts as the second substrate 105 and the nanoprojection array 101 is wrapped around and adhered to the outer surface of the tube 107. A thin PDMS layer was first attached to the outer surface of the tube 107 to act as an adhesive layer. The nanoprojection array 101 was adhered to the PDMS layer. See FIG. 12A. As will be appreciated by those skilled in the art, any suitable method for adhering can be used. FIG. 12B shows an SEM image of the surface of curved tube 107 showing the array of nanoprojections (in this case nanocones 101b).

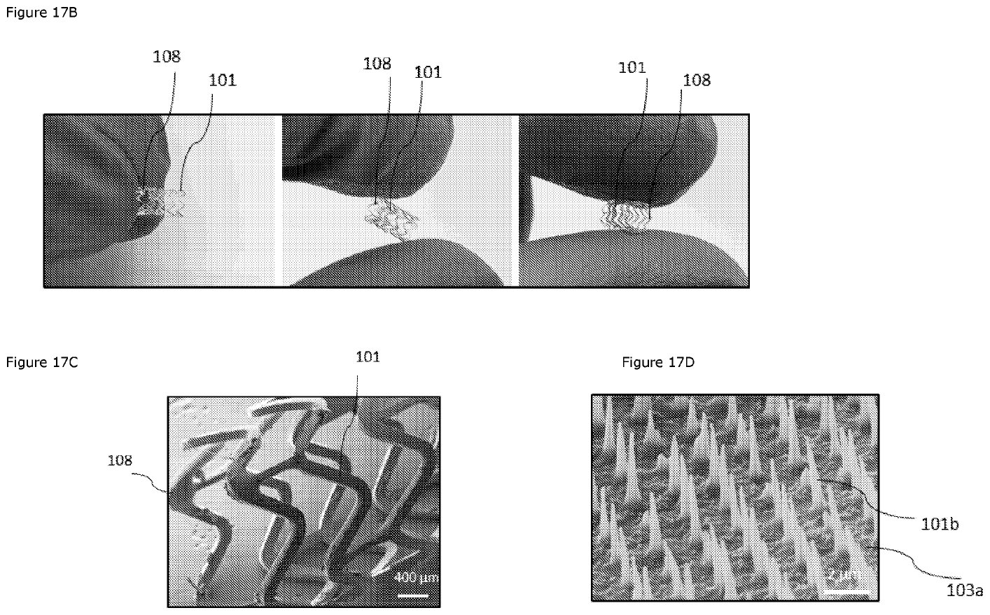

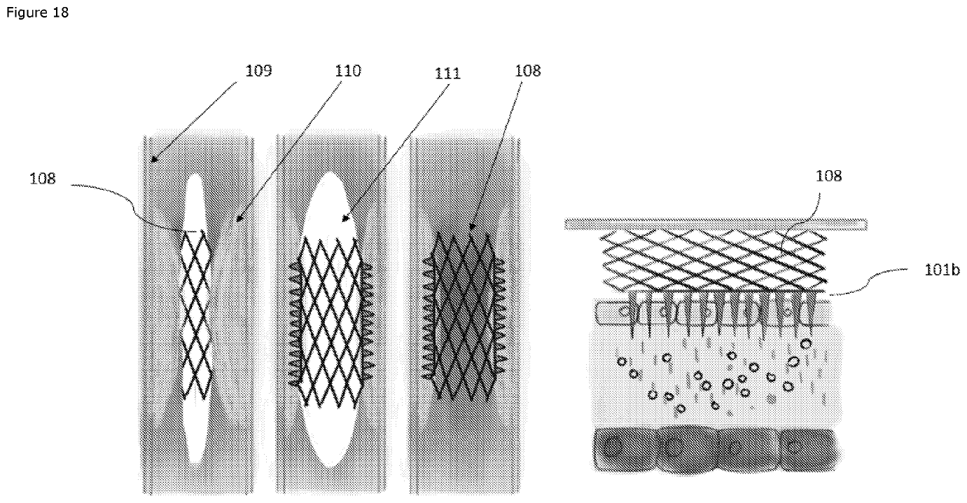

The tube 107 having the nanoprojection array 101 on its outer surface is then formed into a stent 108 using standard machining techniques, including laser cutting, etching, etc. Such methods are well known to those skilled in the art. The derived stent 108 having a nanoprojection array 101 on its outer surface can then be used in angioplasty, namely unblocking arteries and veins. Such a stent 108 is shown schematically in FIG. 17A. An image of a stent 108 having the nanoprojection array on its surface is shown in FIG. 17B. A SEM image of the stent 108 is shown in FIG. 17C. A SEM image of the nanoprojection on the surface of the stent (in this case nanocones) is shown in FIG. 17D, along with the first porous layer 103a. FIG. 18, shows schematically the use of such a stent in angioplasty. The nanoprojections 101b are not shown to scale in FIG. 18.

By integrating the nanoprojection array 101 with the stent 108, the resultant stent configuration helps to improve placement of the stent 108 in the vessel wall 109. In particular, the nanoprojections 101b help fix the stent 108 in position and prevent any movement of the stent 108 relative to the vessel wall 109. In FIG. 18A, the compacted stent 108 is positioned within the vessel 109 at the site of a plaque 110. A balloon 111 is then used to expand the stent 108. On expansion the nanoprojections 101b positioned on the outside surface of the stent 108 abut and penetrate the plaque 110 and/or the vessel wall 109. In addition, when the nanoprojections (e.g., porous nanocones 101b) are loaded with a therapeutic agent, the therapeutic agent can be delivered to the plaque area. See FIG. 18B. Any suitable therapeutic agent can be delivered using the nanoprojection array 101, including agents that prevent or reduce the risk of restenosis.

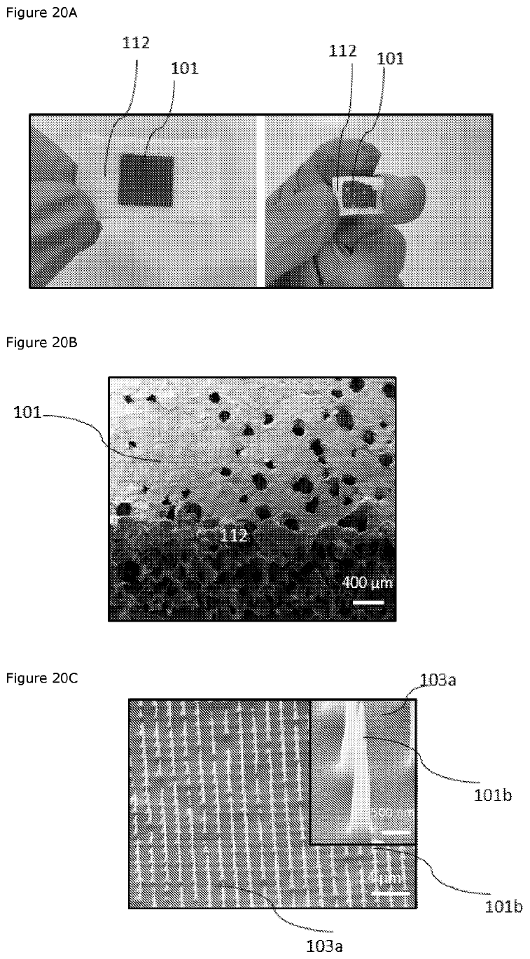

Although the nanoprojection array 101 has been described as being attached to the outer surface of a stent 108, the nanoprojection array 101 can be applied to any medical device that comes into contact with a tissue. For example, the nanoprojection array 101 has been applied to a medical bandage 112. See FIG. 20A. The medical bandage 112 can be used to deliver a therapeutic agent to a tissue when the nanoprojections have been loaded with the therapeutic agent or can be used to obtain a tissue sample. FIG. 20B shows an SEM images of the nanoprojection array 101 on the medical bandage 112. FIG. 20C shows an SEM image of the nanoprojections 101b (in this case nanocones) of array, including the first porous layer 103a, on the medical bandage 112.

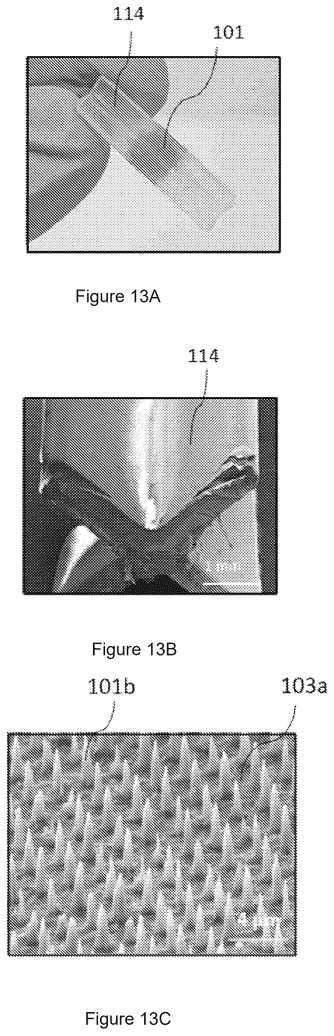

The nanoprojection array can be applied to any device or surface. In FIG. 13, a nanoprojection array was applied to a structure with an X-shaped cross section using the same method as described above with respect to the curved tube (107). FIGS. 13A to 13C show the applied nanoprojection array.

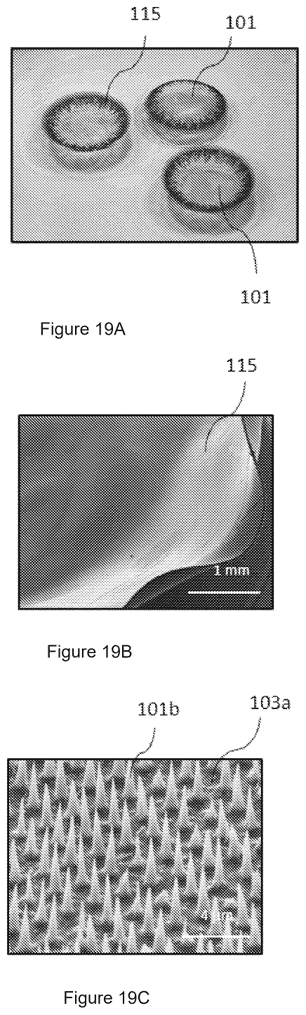

The same method of applying a nanoprojection array as described above was used to transfer a nanoprojection array onto the curved surface of a contact lens (115). See FIG. 19A, wherein contact lenses having the attached nanoprojection array are shown. FIGS. 19B and 19C show SEM images of the nanoprojection array on the contact lens at different magnifications.

The fundamental mechanism and understanding obtained from this transfer approach can be applied to transfer the nanostructure array of the present disclosure onto any substrate with any desired shape.

### Molecular Tissue Sampling and Replica Generation

Another example of an application for a nanoprojection array 101 is in molecular tissue sampling. The analysis of the molecular characteristics of tissues is important to determine the underlying biological processes. In particular, the analysis of the spatial and temporal distribution of a variety of biomolecules in a tissue can provide information that can aid in understanding and treatment of diseases. Many existing methods for sampling molecular tissue require highly invasive procedures which typically require removing biopsy samples or do not enable the determination of the relative spatial distribution of the biomolecules within the tissue.

The inventors have recognised that a nanoprojection array 101 can be used to provide a minimally invasive approach for acquiring a molecular sample from a tissue in order to enable spatiotemporal analysis of the molecular composition of the tissue, which may be used in molecular diagnostics. The inventors have recognised that a minimally invasive approach, as provided by a nanoprojection array 101, enables a molecular tissue replica to be obtained.

FIG. 15 show the key steps involved in an application of a nanoprojection array 101. A nanoprojection array 101 (see inset of FIG. 15), which in this case comprises porous nanocones 101b on a flexible substrate 105, is placed in contact with a target tissue area so that the nanoprojections 101b penetrate the tissue and can absorb molecules present at the target tissue area (see FIG. 15). The topical application of the nanoprojection array 101 to the target tissue area results in the molecules of the target tissue area being absorbed on the nanoprojections 101b and for the relative spatial distribution to be retained. A replica of the molecular composition of the target tissue is thereby obtained. The molecular replica can contain, for example, at least a fraction of the proteins, lipids, metabolites, drugs and nucleic acids present in the target tissue, and their relative localisation is retained. The harvested molecules are intact and can be analysed downstream either in a spatially-resolved manner through imaging approaches known in the art such as spatial transcriptomics, mass spectrometric imaging, imaging mass cytometry, Raman or FTIR imaging (see FIG. 15). Alternatively, the harvested molecules can be analysed following elution (and optionally purification) with homogenous assays such as mass spectrometries (GC-MS, LC-MS, ESI, MALDI), gel electrophoresis, proteomics, transcriptomics, western blotting or qPCR.

FIG. 15 shows that RNA, proteins and lipids can be eluted from the nanoprojection array 101 using known techniques. The methods used to extract the various molecules from the nanoprojection array are set out below.

### 1a. RNA Extraction

All surfaces were wiped with RNase AWAY™ (Thermo Scientific™ 10666421). Biomolecules were extracted with 300 μL TRIzol. To assist the elution of biomolecules from nNs and glass chips a scraper (Falcon® cell scraper, 353085) or ceramic beads (MP Biomedicals, Lysing matrix D, 2 mL tube, 6913050) with 20 s, 4 m/s tissue homogenisation (MP Biomedicals FastPrep-24) were used. The solution was centrifuged at 16000× g for 1 min and the supernatant was collected, to remove the nNs and glass residue. Direct-zol™ RNA MiniPrep Plus (Zymo research, R2070S) was employed for RNA extraction. An equal volume of ethanol 100% was added to the sample before transferring it to a Zymo-Spin™ IIICG Column (Zymo research, C1006). The sample was centrifuged at 16000× g for 30 s, and the flowthrough underwent to protein extraction. The column was transferred to another RNase free tube and underwent 2 pre-washes and 1 wash step using the buffers provided with the columns, as per manufacturer instruction. Finally, the RNA was collected in RNase free water.

### 1b. Total RNA Quantification

Qubit™ RNA HS assay (Thermo Fisher Scientific, Q32852) was employed for total RNA detection, following the user manual from the manufacturer, using a Qubit 3.0 fluorometer (Invitrogen™ Q33216).

### 2a. Protein Extraction

The flowthrough from the RNA extraction in the column was incubated for 30 min on ice, after adding 4 volumes of cold acetone (−20° C.). After centrifugation at 20238 rcf for 10 min, the protein pellet underwent ethanol wash and centrifugation at 20238 rcf for 1 min. The protein pellet was air dried for 10 min at room temperature, then resuspended in 100 μL protein storage buffer: 4 M urea (Sigma-Aldrich, U5128), 1% SDS (Sigma-Aldrich, L3771). Proteins were kept at −80° C. until quantification.

### 2b. Detergent Removal and Desalting

The detergent was removed from the samples in storage buffer by loading 100 μL of sample in pre-dispensed HiPPR™ spin columns (Thermo Scientific™, 88305), then following the user manual from the manufacturer. Desalting was performed by loading 100 μL of sample in Pierce™ Protein Concentrators PES, 3K MWCO (Thermo Scientific™, 88512), following the user manual from the manufacturer. For quantification with CQBCA, the protein concentrator allowed to change the storage buffer to 100 μL of 0.1 M sodium borate (Sigma-Aldrich, HT1002), pH 9.3.

### 2c. Total Protein Quantification

CBQCA protein quantitation kit (Invitrogen™, C6667) was used to increase the sensitivity by 1 order of magnitude, detecting proteins down to 0.1 μg/mL vs 2 μg/mL. A standard curve using bovine serum albumin (Sigma, A9647) was built following the manufacturer instruction. Samples and standard were processed according to the user manual from the manufacturer. Fluorescence was measured using the CLARIOstar® Plus platereader (BGM Labtech), with excitation waveband 465±15 nm and emission waveband 550±20 nm.

### 3a. Lipid Extraction

Biomolecules were extracted with 200 μL CH₂OH—H₂O mixture or CHCl₃. To assist the elution of biomolecules from nNs and glass chips ultrasonication or ceramic beads (MP Biomedicals, Lysing matrix D, 2 mL tube, 6913050) with 20 s, 4 m/s tissue homogenisation (MP Biomedicals FastPrep-24) were used. Samples were immediately placed on ice. The solution was centrifuged at 16000× g for 1 min and the supernatant was collected, to remove the nNs and glass residue. The ratio of CHCl₃—CH₂OH—H₂O was brought to 1:2:0.8 and the sample mixed, then CHCl₃ was added to obtain ratio 2:2:0.8, vortexed for 30 s and allow phase separation. The aqueous layer was removed. The organic phase was transferred into new tubes for quantification.

### 3b. Total Lipid Quantification

A stock solution was prepared for Nile Red (NR) in (9-diethylamino-5H-benzo[α]phenoxazine-5-one, C₂₀H₁₈N₂O₂, Sigma Aldrich 72485) in acetone 1:40 v/v. The samples were dried under N₂ flow to 1/10 of the original volume or until complete evaporation. A volume of 200 μL of water was added to the samples. Immediately after vortexing the samples, 2 μL of NR stock solution was added to the samples. The samples were then vigorously vortexed for 1 min to obtain a microemulsion. The samples were finally transferred to a well plate for analysis. Fluorescence was measured using CLARIOstar® Plus microplate reader, with excitation waveband 530±15 nm and emission waveband 612±100 nm.

The levels of RNA, proteins and lipids that have been eluted from the nanoprojection arrays 101 are shown in FIG. 15D. FIG. 13E shows the results of comparative mass spectroscopy imaging of mouse brain from a histological tissue section and from a molecular replica of the histological tissue section obtained with a nanoprojection array. The tissue and the replica were subjected to unsupervised hierarchical cluster analysis to identify congruent molecular patterns within the tissue section and the replica. The clusters discriminate white matter from grey matter for both tissue section and replica. The average molecular composition of the section and the replica for each white and grey matter are congruent. It can be seen that the replica and the original section are substantially identical indicating that the nanoprojection array can be used to obtain an accurate molecular replica of a tissue.

### Cell Surface

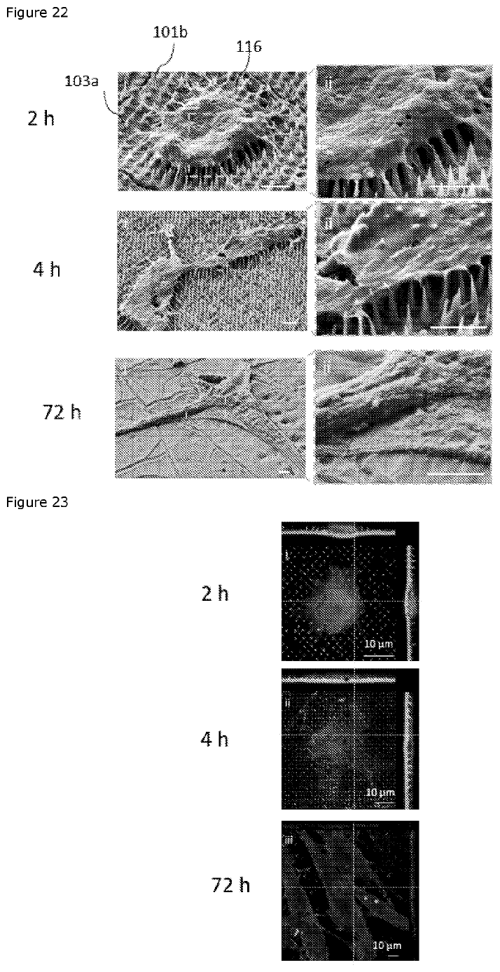

To observe cell behaviour and visualize the cell-nanoneedle interface, human dermal fibroblasts (hdf) cells were seeded over a nanoprojection array attached to a PDMS substrate (obtained using the method described above). The nanoprojection array was imaged by SEM at 2 h, 4 h and 72 h respectively, shown with pseudo colouring in FIG. 22 and confocal in FIG. 23. To seed the hdf cells, the nanoprojection arrays were sterilized with 70% v/v ethanol in deionized water for 1 h, dried and UV irradiated for 20 minutes. The nanoprojection arrays were placed at the bottom of a 24-well plate and rinsed three times with phosphate buffered saline (PBS). Primary human dermal fibroblast (hdf) cells were seeded over the nanoprojections at a density of 1×10⁵ cells/well, and the well plates were kept in the incubator at 37° C. with 1% CO₂. At the desired time points (2 h, 4 h and 72 h) cells on the nanoprojection arrays were fixed using 4% paraformaldehyde (PFA) for 10 minutes, followed by dehydration steps with 30%, 50%, 70%, 90%, 95% and 100% ethanol. In FIGS. 22 and 23 it can be seen that 2 h after seeding the tip of the nanoneedles interface with the cytosol and are close to the nucleus, and that the cells retained the spherical shape of the early stages of cell spreading. As time progressed, cells adhered and spread, wrapping the tips of their filopodia around the neighbouring nanoprojections. Throughout the process of interfacing the nanoprojections experienced progressive degradation, and at 72 h, nanoprojections were fully degraded without any recognizable nanoprojections remaining. As indicated previously, the rate of biodegradation of the nanoprojections can be tailored.

While the above disclosure provides some example applications of nanoprojection arrays, nanoprojection arrays can also be used in other applications such as in sensors, microscopy, biomedical instruments for nanosurgery and topical gene therapy. Furthermore, the values provided for the different empirically measured parameters, such as the parameters for the different etches, throughout the description, are ‘approximate’ values in that the values are measured within the bounds of quality/tolerances of the measurement instrumentation.

1. The method of forming an array of nanoprojections, the method comprising:
providing a first substrate;
performing a first etch of the first substrate to form an array of nanoprojections on the first substrate;
performing a second etch of the first substrate to form a first porous layer in the first substrate, wherein the first porous layer underlies the array of nanoprojections, and to either:
(a) form a second porous layer in the first substrate, wherein the second porous layer underlies the first porous layer and has a higher porosity than the first porous layer such that the first porous layer is detachable from the second porous layer; or
(b) remove a layer of the first substrate underlying the first porous layer so that the first porous layer is detachable from the first substrate;
attaching an adhesive layer over the top of the array comprising the nanoprojections so that the adhesive layer adheres to the free ends of the nanoprojections, and separating the adhesive layer, the array of nanoprojections and the first porous layer away from the second porous layer or the first substrate; and
attaching the array of nanoprojections and the first porous layer to a second substrate so that the first porous layer is adjacent to the second substrate and between the second substrate and the array of nanoprojections.

2. The method of forming an array of nanoprojections according to claim 1, wherein performing the first etch and/or the second etch provides porous nanoprojections.

3. The method of forming an array of nanoprojections according to claim 1, wherein a mask is provided on the surface of the first substrate in a pattern which enables selective etching of the first substrate to form the array of nanoprojections.

4. The method of forming an array of nanoprojections according to claim 3, wherein the mask comprises an array of nanodots.

5. The method of forming an array of nanoprojections according to claim 4, wherein the array of nanodots comprises dielectric nanodots.

6. The method of forming an array of nanoprojections according to claim 1, wherein areas of the first substrate that are to be etched have a metal layer deposited thereon.

7. The method of forming an array of nanoprojections according to claim 6, wherein performing the first etch comprises performing a metal-assisted chemical etch (MACE) to etch the first substrate to form the array of nanoprojections.

8. The method of forming an array of nanoprojections according to claim 1, wherein the second etch comprises a multi-step wet etch.

9. The method of forming an array of nanoprojections according to claim 8, wherein performing the second etch comprises performing an electrochemical etch which comprises a first etch step at a first set of bias conditions for forming the first porous layer in the first substrate, and a second etch step at a second set of bias conditions, for either (a) forming the second porous layer in the first substrate, or (b) removing the layer of the first substrate underlying the first porous layer.

10. The method of forming an array of nanoprojections according to claim 9, wherein the first etch step has a longer duration and/or a lower bias current than the second etch step.

11. The method of forming an array of nanoprojections according to claim 1, wherein the first substrate comprises a silicon substrate.

12. The method of forming an array of nanoprojections according to claim 11, wherein the etchant for the second etch comprises hydrofluoric acid.

13. The method of forming an array of nanoprojections according to claim 12, wherein the etchant for the second etch further comprises ethanol.

14. The method of forming an array of nanoprojections according to claim 1, wherein the adhesive layer is soluble.

15. The method of forming an array of nanoprojections according to claim 14, wherein the method further comprises removing the adhesive layer by solubilisation after separating the adhesive layer, the array of nanoprojections and the first porous layer away from the second porous layer or the first substrate.

16. The method of forming an array of nanoprojections according to claim 1, wherein the method additionally comprises performing a third etch on the array of nanoprojections so that the nanoprojections are shaped as nanocones.

17. The method of forming an array of nanoprojections according to claim 16, wherein the third etch is performed before separating the first porous layer away from the second porous layer or the first substrate.

18. The method of forming an array of nanoprojections according to claim 16, wherein the third etch is performed after separating the first porous layer away from the second porous layer or the first substrate.

19. The method of forming an array of nanoprojections according to claim 16, wherein performing the third etch comprises performing a dry etch.

20. The method of forming an array of nanoprojections according to claim 19, wherein the dry etch comprises a reactive-ion etch.

21. The method of forming an array of nanoprojections according to claim 1, wherein the second substrate is configured to be a transparent and/or a flexible substrate.

22. The method of forming an array of nanoprojections according to claim 1, wherein the second substrate is a hydrogel substrate.

23. The method of forming an array of nanoprojections according to claim 1, wherein the second substrate is a polymer substrate, a bandage, a metal or metal oxide substrate, a ceramic substrate, a glass substrate, a silicon substrate, a quartz substrate, a diamond substrate, or a carbon substrate.

24. The method of forming an array of nanoprojections according to claim 1, wherein the second substrate is a surface of a medical device.

25. An array of nanoprojections formed using the method of claim 1.

26. The array of nanoprojections according to claim 25, wherein the nanoprojections are biodegradable.

27. The array of nanoprojections according to any of claim claim 25, wherein the nanoprojections are loaded with one or more therapeutic or diagnostic agents.

28. A method of administering one or more therapeutic or diagnostic agents to cells and/or a tissue comprising placing the array of nanoprojections according to claim 27 in contact with the cells and/or tissue.

29. A method of obtaining a molecular sample from cells or a tissue, comprising placing an array of nanoprojections in contact with the cells or tissue so that molecules present within the cells or tissue are absorbed onto the nanoprojections of the array.

30. A method of obtaining a molecular sample from cells or a tissue, comprising placing an array of nanoprojections in contact with the cells or tissue so that molecules present within the cells or tissue are absorbed onto nanoprojections of the array, wherein the array of nanoprojections is the array of nanoprojections of claim 25.

31. A medical device that in use comes into contact with the body of a patient, wherein the medical device has a nanoprojection array according to claim 25 on at least part of its surface.

32. A device comprising:
an array of nanoprojections formed on a first porous layer, wherein the nanoprojections are at least partially porous; and
a second substrate wherein first porous layer is attached to the second substrate so that the first porous layer is adjacent to the second substrate and between the second substrate and the array of nanoprojections, and wherein the first porous layer and the second substrate are formed from different materials.

33. A method of forming an array of nanoprojections, the method comprising:
providing a first substrate;
performing a first etch of the first substrate to form an array of nanoprojections on the first substrate;
performing a second etch of the first substrate to form a first porous layer in the first substrate, wherein the first porous layer underlies the array of nanoprojections and forming a second porous layer in the first substrate, wherein the second porous layer underlies the first porous layer and has a higher porosity than the first porous layer such that the first porous layer is detachable from the second porous layer.
